(19) United States
(12) Patent Application Publication
(10) Pub. No.: US 2005/0003016 A1
(43) Pub. Date: Jan. 6, 2005

(54) Controlled release polymersomes

(76) Inventors: Dennis E. Discher, Philadelphia, PA (US); Fariyal Ahmed, Philadelphia, PA (US)

Correspondence Address: DILWORTH PAXSON LLP, 3200 MELLON BANK CENTER, 1735 MARKET STREET, PHILADELPHIA, PA, 19103, US

(21) Appl. No.: 10/812,292

(22) Filed: Mar. 29, 2004

Related U.S. Application Data
(63) Continuation-in-part of application No. 09/460,605, filed on Dec. 14, 1999.
(60) Provisional application No. 60/459,049, filed on Mar. 28, 2003.

(51) Int. Cl. A61K 9/127; B01J 13/02; B01J 13/04

(52) U.S. Cl. 424/490; 264/4.1

(57) ABSTRACT

The present invention provides methods for preparing stable, purely synthetic, self-assembling, controlled release, polyethylene oxide (PEO)-based polymersome vesicles, and the resulting PEO-based polymersomes capable of such controlled release, and methods of use therefor for the controlled transport and delivery of encapsulatable active agents contained therein. Further provided are methods for controlling destabilization of the vesicle membrane and the resulting hydrolysis-triggered, controlled release of active agent(s) encapsulated in the vesicle by controlling the blend ratio (mol %) of hydrolysable PEO-block copolymer of the hydrophilic component(s) and of the more hydrophobic PEO-block copolymer component(s) to produce amphiphilic high molecular weight PEO-based polymersomes, wherein the PEO volume fraction (f_{EO}) and chain chemistry control encapsulant release kinetics from the copolymer vesicles and the polymersome carrier membrane destabilization.

### CROSS REFERENCE TO RELATED APPLICATIONS

This application is a Continuation-in-Part application of U.S. patent application Ser. No. 09/460,605, filed Dec. 14, 1999, and also claims priority to U.S. Provisional Application No. 60/459,049 filed Mar. 28, 2003, which is incorporated herein in its entirety.

### GOVERNMENT SUPPORT

This work was supported in part by a grant from the National Institutes of Health, grant number R21. The government may have certain rights in this invention.

### FIELD OF THE INVENTION

The present invention relates to hydrolysis-triggered controlled release vesicles and supporting encapsulation studies.

### BACKGROUND OF THE INVENTION

Membranes that are stable in aqueous media are heavily relied upon for compartmentalization by biological cells. A biomembrane also possesses stability and other thermo-mechanical properties which, in addition to biocompatibility, affect how lipid vesicles, liposomes, that are assembled in vitro, can effectively encapsulate and deliver a long list of bioactive agents (Needham et al., in Vesicles, M. Rosoff, Ed. (Dekker, New York, 1996), chap. 9; Cevc & Lasic in Handbook of Biological Physics, chaps. 9-10, 1995; Koltover et al., Science 281:78 (1998); Harasym et al., Cancer Chemother. Pharmacol. 40:309 (1997)). The typical liposome is comprised of one or more bilayer membranes, each approximately 5 nm thick and composed of amphiphiles such as phospholipids. Each bilayer exists as a temperature- and solvent-dependent lamellar phase that is, in its surface, in a liquid, gel, or liquid-gel coexisting state. Because of a certain intrinsic biocompatibility of phospholipid vesicles, many groups have developed them for use as encapsulators and delivery vehicles. Most, if not all, conventional liposome systems have proven to be both inherently leaky (Lasic et al., Medical Applications of Liposomes, Elsevier, Amsterdam, New York, 1998, pp. 1-16) and short-lived in the circulation (Liu et al., Biochim. Biophys. Acta. Biomembranes 1235:140-146 (1995)). Vesicles surrounded by a lipid bilayer can range in diameter from as small as tens of nanometers to giants of 0.5-40 microns.

Phospholipid vesicles are materially weak and environmentally sensitive. Transit through the digestive tract, for example, can expose liposomes to a host of solubilizing agents. Repeated transit through the microcirculation can also tear apart giant phospholipid vesicles that cannot withstand high fluid shear. Smaller phospholipid vesicles may not fragment, but they tend to adhere, and are thus cleared from circulation. Circulating cells suppress their own adhesion partly through a brushy biopolymer layer, known as the glycocalyx, which faces the environment. The glycocalyx has, to some extent, been mimicked in liposome systems by the covalent addition to lipids of hydrophilic polyethyleneglycol (PEG) polymer chains. To maximally extend a vesicle's circulation lifetime (about ten hours), a suitable PEG weight is added, ranging between about two and five kilograms/mole.

Past efforts to enhance the stability of lipid lamellae against shear and other factors, resulted in the synthesis of many different modified lipid molecules with polymerizable double bonds. Such bonds were located either at the surfactant head group, or more commonly, at different locations on the hydrophobic tails (Fendler et al., Science 223:888 (1984); Liu et al., Macromolecules 32:5519 (1996)). This approach clearly had the ability to generate covalently inter-connected poly-amphiphiles when reacted after self-assembly into membranes per ordinary lipids. However, a fully, covalently interconnected network of lipids requires complete cross-linking of the membrane of a vesicle, and the full extent of cross-linking achievable with cross-linkable lipids appears to be difficult to ascertain. O'Brien's group (Sisson et al., Macromolecules 29:8321 (1996)) has used solubility in hexafluoropropanol to estimate a degree of polymerization up to at least 1000. This corresponds to a vesicle diameter of about 10 nanometers, if one assumes complete cross-linking within and between layers of the bilayer, and a typical lipid area of about 0.5 square nanometers per lipid. Detergent induced leakage of entrapped solutes was strongly inhibited by cross-linking. It is clear, however, that no fully cross-linked lipid vesicle larger than several hundred nanometers has been reported.

Systems based on chemically active monomers, such as phospholipase sensitive monomers (Jorgensen et al., FEBS Lett. 531:23-27 (2002); Davidsen et al., Biochim. Biophys. Acta 1609:95-101 (2003)) or pH/light destabilized lipids (Gerasimov et al., Biochim. Biophys. 1324:200-214 (1997); Wymer et al., Bioconjugate Chemistry 9:305-308 (1998); Boomer, et al., Chemistry and Physics of Lipids 99:145-153 (1999); Adlakha-Hutcheon et al., Nature Biotechnology 17:775-779 (1999)), and polyethyleneglycol (PEGs lipids (Kirpotin et al., FEBS Lett. 388:115-118 (1996); Zalipsky et al., Bioconjugate Chemistry 10:703-707 (1999); Shin et al., J. Controlled Release 91:187-200 (2003); Boomer et al., Langmuir 19:6408-6415 (2003); Bergstrand et al., Biophysical Chemistry 104:361-379 (2003)) have been introduced as a means to control drug release. As stabilizers, a small percentage (5-10%) of PEG-lipid was found, some time ago, to also delay liposome clearance [14]. In other words, PEG imparts stealthiness. However, above 5-10%, PEG-lipid destabilizes the vesicle or dissociates from it.

Many wholly synthetic, amphiphilic molecules are significantly larger (in molecular weight, volume, and linear dimension) than phospholipid amphiphiles, and have therefore been called “super-amphiphiles” (Cornelissen et al., Science 280:1427 (1998)). Cornelissen et al. used polystyrene (PS) as a hydrophobic fraction in their series of synthetic block copolymers designated PS40-b-(isocyano-L-alanine-L-alanine)y. For y=10, but not y=20 or 30, small collapsed vesicles with diameters ranging from tens of nanometers to several hundred, and a bilayer thickness of 16 nanometer were mentioned as existing under a single acidic buffer condition (0.2 mM Na-acetate buffer, pH 5.6). However, bilayer filaments and superhelical rods existed, without explanation, under the same solution conditions, thus making the stability of the collapsed vesicles, relative to the other microstructures, highly uncertain for the studied polymer. Furthermore, no demonstration of semi-permeability was reported, and reasons for apparent vesicle collapse were not given, further raising questions of vesicle stability.

Additional spherical shell structures smaller than a few hundred nanometers, and which required the presence of organic solvents mixed into water to drive their formation, include those assembled from various block copolymers as observed by Yu et al., Macromolecules 31:1144 (1998); Ding et al., J. Phys. Chem. B 102:6107 (1998); Henselwood et al., Macromolecules 31:4213 (1998)). However, only Cornelissen et al., 1998, reported constructing a wholly synthetic super-amphiphile having the capacity to self-assemble in aqueous solution, albeit only under moderately acidic pH conditions, into a vesicle-like microstructure.

Both amphiles and super-amphiphiles can exist in a broad variety of microphases. Based on the work of Hajduk et al. (see, J. Phys. Chem. B 102:4269 (1998)), the ability of super-amphiphilic block copolymers to form lamellar phases in aqueous solutions can be regulated by both synthetic tuning of polymer chemistry and physical variables like, such as concentration and temperature. Evidence has now accumulated that in dilute solutions certain diblock copolymers, such as polyethyleneoxide-polyethylethylene (PEO-PEE, wherein PEO is structural equivalent to PEG), can form not only worm-like micelles (Won et al., Science 283:960-963 (1999)), but also unilamellar vesicles (Discher et al., Science 284:1143 (1999)).

In addition, because of the synthetic control over molecular composition, properties of membranes assembled from super-amphiphiles can be controlled in novel ways. For instance, a super-amphiphilic polymer can be made far more reactive than a much smaller phospholipid molecule simply because more reactive groups can be designed into the polymer. The principle was first illustrated for the aforementioned worm-like micelles in which polyethyleneoxide-polybutadiene (PEO-PBD) mesophases were successfully cross-linked into bulk materials with completely different properties, notably an enhanced shear elasticity (Won et al., 1999). The resulting microstructures, though assembled in water, could withstand dehydration, as well as exposure to an organic solvent, such as chloroform. In the absence of cross-linking, microstructures of amphiphiles and super-amphiphiles are generally unstable to treatments that could otherwise prove very useful for a range of applications that might benefit from, for example, sterilization, or long-term dry storage.

Despite recent advances, there remained until the present invention a long felt need in the art for methods to control the release of one or more active agents encapsulated within stable, aqueous-formed vesicles which could be more broadly engineered, but still have demonstrable features in common with a biomembrane or a mimic, including: biocompatibility, selective permeability to solutes, the ability to retain internal aqueous components and control their release, the ability to deform yet be relatively tough and resilient, and the ability to extensively cross-link within the membrane in order to withstand extreme environments. Although PEG-lipid is useful for some degree of stealthiness, the question remained unanswered as to how to achieve greater stealthiness and gain selective control of release.

### SUMMARY OF THE INVENTION

The present invention meets the need in the art by providing not only an illustrative set of stable super-amphiphilic vesicles in biocompatible, aqueous solutions, but it also provides vesicles which are entirely synthetic, creating an opportunity to tailor the dynamics, structure, rheological and even optical responses of the membrane based on its composition. The polymer vesicles of the present invention are called “polymersomes.” Analogous to “liposomes” made from phospholipids, the material properties of the polymersome vesicles can be readily measured using techniques that have been largely developed for phospholipid vesicles and biological cells. Furthermore, the ability to cross-link the polymer building blocks affords a novel opportunity to provide mechanical control and stability to the vesicle on the order of that which is provided by the protein skeleton in the plasma membrane of a cell.

Polymersomes of the present invention possess membranes capable of self-repair, adaptability, portability, resilience, and are selectively permeable, thereby providing, for example, long-term, reliable and controllable vehicles for the delivery or storage of drugs or other compositions, such as oxygen, to the patient via the bloodstream, gastrointestinal tract, or other tissues, as replacement artificial tissue or soft biomaterial, as optical sensors, and as a structural basis for metal or alloy coatings to provide materials having unique electric or magnetic properties for use in high-dielectric or magnetic applications or as microcathodes.

In accordance with the present invention, to provide greater control over release of an encapsulant than that which is possible simply by the inclusion of PEG lipids in the carrier, there are provided vesicles comprising semi-permeable, thin-walled encapsulating membranes, wherein the membranes are formed in an aqueous solution, and wherein the membranes comprise one or more synthetic super-amphiphilic molecules. The invention relates to all super-amphiphilic molecules, which have hydrophilic block fractions within the range of 20-50% by weight, and which achieve some or all of the above capsular states of matter.

Further provided are vesicles and encapsulating membranes, wherein at least one super-amphiphile molecule is a block copolymer, and wherein the resulting vesicle is termed a polymersome. The thus provided polymersomes may be comprised of multi-block copolymers, most preferably, but not limited to diblock or triblock copolymers. Moreover, in certain preferred embodiments of the present invention are provided polymersomes in which all of the super-amphiphile molecules are block copolymers. The block copolymers useful in the present invention may be selected from any known block copolymer, including, for example polyethylene oxide (PEO), poly(ethylethylene) (PEE), poly(butadiene) (PB or PBD), poly(styrene) (PS), and poly(isoprene) (PI). As needed, monomers for these polymers will be denoted by EO, EE, B or BD, S, and I, respectively.

In addition the present invention provides polymersomes, wherein the vesicles are capable of self-assembly in aqueous solution.

The present invention also provides methods for the preparation of mixtures of super-amphiphiles from smaller amphiphiles, such as phospholipids up to at least 20% mole fraction, which have also been shown capable of integrating into stable encapsulating membranes.

Further provided in the present invention are reactive amphiphiles that can be covalently cross-linked together, over a many micron-squared surface, while maintaining semi-permeability of the membrane. Cross-linked polymersome are characterized as having the ability to withstand exposure to organic solvents, boiling water, dehydration and rehydration in an aqueous solution without visibly or significantly affecting the integrity of the membrane.

In addition, the present invention provides polymersomes, wherein the vesicle is biocompatible. Further provided are vesicles for the retention, delivery, and/or extraction of materials, which may require membrane biocompatibility and may or may not take advantage of the novel thermal, mechanical, or chemical properties of the surrounding membranes.

The present invention also provides polymersomes which encapsulate one or more “active agents,” which include, without limitation compositions such as a drug, therapeutic compound, dye, nutrient, sugar, vitamin, protein or protein fragment, salt, electrolyte, gene or gene fragment, product of genetic engineering, steroid, adjuvant, biosealant, gas, ferrofluid, or liquid crystal. The thus “loaded” polymersome may be further used to transport an encapsulatable material (an “encapsulant”) to or from its immediately surrounding environment.

Moreover, the present invention provides methods of using the polymersome or encapsulating membrane to transport one or more of the above identified compositions to or from a patient in need of such transport activity. For example, the polymersome could be used to deliver a drug or therapeutic composition to a patient's tissue or blood stream, or it could be used to remove a toxic composition from the blood stream of a patient with, for example, a life threatening hormone or enzyme imbalance.

Also provided by the present invention are methods of preparing an “empty” polymersome, wherein the preferred methods of preparation include at least one step consisting of a film rehydrating step, a bulk rehydrating step, or an electroforming step.

Further provided are methods for controlling the release of an encapsulated material from a polymersome by modulating and controlling the composition of the membrane. For example, one preferred method of controlling the release of an encapsulated material from a polymersome or encapsulating membrane entails cross-linking the membrane. In another preferred method, release of the encapsulated material is controlled by forming the encapsulating membrane from at least one cross-linkable amphiphile and at least one non cross-linkable molecule, followed by subjecting the thus destabilized membrane to chemical exposure or to waves of propagated light, sound, heat, or motion.

In addition, the present invention provides methods for controlling release of an active agent from hydrolysis-triggered controlled release polymer vesicles. Particularly useful are diblock copolymers, such as biomedically acceptable copolymers, including without intended limitation, polyethyleneglycol-poly-L-lactic acid (PEG-PLA) or polyethyleneglycol-polycaprolactone (PEG-PCL). Rates of encapsulant release from the hydrolysable vesicles are accelerated with an increased proportion of PEG, but are delayed in the presence of more hydrophobic chain chemistry (i.e., PCL). Contrary to the known uses of PEG lipids to impart stealthiness, there are no previously known compositions in which the acyl chains (hydrophobic part) of the PEG lipid degrades, or in which the PEG chain is designed to degrade to trigger the controlled release of an encapsulant. Using polyesters to achieve controlled release is not known, primarily because polyesters are oxygen-rich. Therefore, only when a polyester chain is made long enough, as in the present invention, will it be sufficiently hydrophobic to drive self-assembly; albeit blending, as well as vesicle assembly, both require that the chains not be so large that copolymers separate during vesicle formation.

In addition, rates of release of an encapsulant rise linearly with the molar ratio of a second degradable copolymer which is also blended into the membranes, that is, a non-degradable, PEG-based block copolymer, such as, but not limited to, PEG-polybutadiene (PBD). With all compositions, in both 100 nm and giant vesicles, the average release time (from hours to days) reflects a highly quantified process in which any given vesicle is either “intact,” thereby retaining its encapsulant, or its membrane is “porated” and slowly disintegrates. Poration occurs as the hydrophobic PLA or PCL block is hydrolytically scissioned, progressively generating an increasing number of pore-preferring copolymers in the membrane. Kinetics of this evolving detergent mechanism overlay the phase behavior of amphiphiles with transitions from membranes to micelles allowing controlled release.

Thus, provided are methods for preparing stable, purely synthetic, self-assembling, controlled release, polyethylene glycol (PEG)-based polymersome vesicles having a semi-permeable, thin-walled encapsulating membrane and at least one hydrophilic active agent encapsulated therein, wherein the method comprises determining the appropriate blend ratio (mol %) of the hydrophilic and the non-hydrophilic copolymer components that will produce PEG-based polymersomes having a desired controlled release rate of the hydrophilic encapsulant; selecting at least one polyester to effect the desired ratio for polyester chain hydrolysis (f_{EO}), thereby controlling encapsulant release kinetics and polymersome carrier membrane destabilization; and blending in aqueous solution at least one hydrophilic, hydrolytically-degradable, hydrophilic block copolymer with at least one inert, non-hydrophilic block copolymer to produce PEG-based polymersomes having the desired controlled release rate of hydrophilic or hydrophobic encapsulants contained therein.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings. It should be understood, however, that the invention is not limited to the precise arrangements and instrumentalities shown.

FIG. 1 depicts the molecular assemblies and copolymer structures in water. FIG. 1A is a schematic representation of diblock copolymer EO₄₀-EE₃₇ The number-average molecular weight is ˜3900 g/mol. For a simple comparison of relative hydrophobic core thickness d, a typical lipid bilayer is schematically shown next to the assembly of copolymers. FIG. 1B depicts aqueous suspensions of EO₄₀-EE₃₇ vesicles in dominant co-existence with rod-like (black arrow) and spherical (gray arrow) micelles. Observations were made by cryo-TEM. The scale bar at lower left is 20 nm and the mean lamellar thickness is ˜8 nm with very little variation, consistent with unilamellar vesicles.

FIG. 2 depicts giant unilamellar vesicles of EO₄₀-EE₃₇. FIG. 2A depicts a vesicle immediately after electroformation in 100 mM sucrose solution. FIG. 2B depicts encapsulation of 10-kD Texas Red-labeled dextran. FIGS. 2C and 2D depict the microdeformation of a polymersome. The arrow marks the tip of an aspirated projection as it is pulled by negative pressure, ΔP, into the micropipette. As shown, aspiration acts to (i) increase membrane tension, τ=1/2ΔPRₚ(1−Rₚ/Rₛ), where micropipette Rₚ and Rₛ are the respective radii of the micropipette and the outer spherical contour; and (ii) expand the original, projected vesicle surface area, Aₒ, by the increment ΔA.

FIG. 3 graphically depicts the mechanical properties of polymersome membranes as assessed by micromanipulation. FIG. 3A shows membrane elasticity in terms of membrane tension versus area expansion. Filled circles indicate aspiration; open circles indicate graded release. The upper left inset shows the distribution of measurements for the bending modulus, K_{b}, as obtained from the initial phase of aspiration. The lower right inset shows the distribution of measurements for the area expansion modulus, Kₐ, as obtained from the linear phase of aspiration. FIG. 3B shows membrane toughness as determined by aspiration to the point of rupture (asterisk). For comparison, aspiration to the point of rupture of an electroformed 1-stearoyl-2oleoyl phosphatidylcholine (SOPC) lipid vesicle is also shown.

FIG. 4 depicts shape transformations driven by osmotic swelling of a single polymersome as imaged by phase contrast video microscopy. The vesicle was formed in 100 mOsm sucrose, and the external sucrose solution was progressively diluted with distilled water from ˜150 mOsm glucose over a period of 90 min. The transformation is shown as a progression beginning with FIG. 4A, which shows a giant tubular state that swells with the initial appearance of interconnected spheres that conserve vesicle topology, shown in FIGS. 4B through 4C and inset. This is followed by the coalescence and disappearance of the small spheres, a form of Ostwald ripening (FIGS. 4D through 4E) before final transformation to a single, tensed sphere (FIG. 4F). The entire swelling sequence is predicated on the vesicle's non-zero permeability to water accompanied by impermeability to the entrapped sucrose solute.

FIG. 5 indicates thermal and physiological solution stability of EO₄₀-EE₃₇ vesicles. FIG. 5A shows the membrane's area expansion with increasing temperature, and its stability at 37° C. The vesicle is held at a fixed membrane tension of less than 4 mN/m. Relative polymer vesicle area, α, is shown against temperature. The overall thermal expansivity is approximately 1.9×10⁻³ per degree C. FIG. 5B demonstrates the long-term stability of polymersomes in phosphate buffered saline (PBS).

FIG. 6 shows a Texas Red-phosphatidylethanolamine (PE) lipid probe uniformly integrated into EO₄₀-EE₃₇ vesicles. FIG. 6A shows the uniformity of fluorescence (3 mol %) around an aspirated contour of membrane. The radius of the pipette is about 2.5 microns. FIG. 6B shows that the contour intensity increases linearly up to about 10 mol % Texas Red PE.

FIG. 7 demonstrates the encapsulation of globular proteins. FIG. 7A shows a 15 μm polymersome encapsulating myoglobin. FIG. 7B shows a 5 μm polymersome encapsulating hemoglobin. FIGS. 7C and 7D show a 25 μm polymer vesicle containing fluorescein-tagged bovine serum albumin (BSA) encapsulated at 0.5 g/l 24 hours earlier and viewed in phase contrast (FIG. 7C) and fluorescence (FIG. 7D), respectively.

FIG. 8 depicts a biocompatibility test in which both red cells and polymersomes were suspended in 250 mOsm phosphate buffered saline in an opened chamber to determine cell adhesion. A polymersome was manipulated by a micropipette (Rₚ=2 μm) into contact with a granulocyte. Initial contact at time point 0 is shown in FIG. 8A. FIGS. 8B and 8C depict the complete lack of activation of the white cell (which would be observed as extension of pseudopods) or adhesion between the cells at time points 62 and 63 seconds, respectively, after initial contact.

FIG. 9 depicts phase contrast images of unilamellar, 15 microns vesicles of EO₂₆-BD₄₆ with corresponding schematic representations of the membrane before the cross-linking reaction, wherein the osmotically inflated vesicles are spherical (FIG. 9A); and after the cross-linking reaction (FIG. 9C). FIG. 9B depicts a fluid phase vesicle, which has been osmotically deflated, resulting in a flaccid shape, but maintaining a smooth contour. By comparison, FIG. 9D depicts a solid-like, cross-linked membrane, which has been osmotically deflated, resulting in a flaccid shape which is not smooth.

FIG. 10 depicts the stability of an EO₂₆-BD₄₆ vesicle in chloroform. FIG. 10A depicts a vesicle in aqueous solution being pulled into a micropipette (Rₚ=4.5 μm) by negative pressure, ΔP. FIG. 10B depicts the same vesicle imaged immediately after being placed into chloroform. No noticeable change was observed in the vesicle after 30 minutes exposure to the chloroform (FIG. OC), or after return of the vesicle back into the aqueous solution (FIG. 10D).

FIG. 11 depicts the dehydration of a vesicle upon exposure to air. FIG. 11A depicts a vesicle in aqueous solution pulled into a micropipette (Rₚ=3.5 μm) by negative pressure, ΔP. FIG. 11B depicts the same vesicle imaged within seconds after its removal from the aqueous solution and exposure to the air. By comparison, as depicted in FIG. 11C, rehydration occurs immediately upon reinsertion of the same vesicle back into the aqueous solution. The original shape is nearly restored within 1 minute, as depicted in FIG. 11D, indicating the retention of solutes.

FIGS. 12A and 12B illustrate the mechanical properties of the cross-linked polymersomes. FIG. 12A is the micropipette aspiration curve for a single, initially flaccid and smooth contour vesicle pulled to a length L into a micropipette. Rₚ is the micropipette radius. At high aspiration pressures, the vesicle interior becomes hydrostatically pressurized. The reversible, initial slope of such a curve is plotted, for a total of ten vesicles, against Rᵥₑₛ/Rₚ in FIG. 12B. This initial slope vanishes in the limit of Rᵥₑₛ =Rₚ, and, above this, resistance to aspiration increases linearly with Rᵥₑₛ/Rₚ. The slope of the fitted line provides an estimate of the membrane's elastic shear modulus (μ) which is independent of vesicle size and which is a property arising only with cross-linking.

FIGS. 13A-13C depict decreased stability of the vesicle fabricated from mixtures of EO₂₆-BD₄₆ and EO₄₀-EE₃₇. FIG. 13A shows 60:40 EO₂₆-BD₄₆: EO₄₀-EE₃₇ vesicle after the cross-linking reaction was completed. FIGS. 13B and 13C show the same vesicle aspirated into a micropipette (Rₚ=1.5 μm) by negative pressure, ΔP=2 cm of water, and ΔP=10 cm of water, respectively. The increased pressure in FIG. 13C leads to perforation of the membrane and leakage of its contents.

FIGS. 14A-14D depict copolymer proportions, resulting architectures, and preliminary drug loading capabilities. FIG. 14A is an illustration of diblock copolymer chains as a function of PEG (or PEO) volume fraction, f_{EO}. As shown, increasing the f_{EO} fraction (e.g., degrading the length of the hydrophobic block) induces a molecular-scale transition: a bilayer forming copolymer f_{EO}˜0.25-0.42) eventually transforms into a membrane-lytic cone-shaped detergent (f_{EO}>0.5). FIG. 14B provides two cryo-TEM images of morphologies exhibited by diblock copolymers, as a self-assembled vesicle of PEG-PLA diblock copolymer OL1, and as several worm-like and spherical aggregates of the inert block copolymer of hydrogenated PEG-PBD. Scale bar is ˜20 nm. FIG. 14C provides two fluorescent images of giant architectures in dilute solution. The PLA block of OL1 is labeled, thus showing vesicles comprising fluorescently labeled OL1 blended with the unlabelled PEO-PBD copolymer, OB18. Intensity analysis (inset) of the fluorescent vesicles demonstrates edge brightness, and localization of OL1 in the vesicle membrane. As shown, at later times, blends also exhibit worm-like micelle morphologies. FIG. 14D shows doxorubicin loaded vesicles imaged by fluorescence. Scale bars are 8 μm.

FIGS. 15A and 15B graphically depict block copolymer blend miscibility in giant vesicles. FIG. 15A shows a proportional increase in membrane fluorescence with increased mol % of fluorescent TMRCA-OL2 in a blended polymersome membrane. Based on the strong intensity with 4 mol % (unfilled white star) of fluorescent OL2, this mol % was used in all further studies of blends. FIG. 15B shows a proportional increase of membrane fluorescence intensity with increasing OL2 (total) in OL2/OB18 blended polymersomes. In each figure, n>10 vesicles (unless indicated) of diameter 2-6 μm were analyzed by fluorescence microscopy under conditions of constant dilution (1:50), and fixed camera gain and exposure time.

FIGS. 16A and 16B depict release from polymer vesicles. FIG. 16A shows phase contrast microscopy images of degradable polymersome carriers in a sealed chamber. Vesicles of 25 mol % blends of OL1 in OB18 are loaded with sucrose (300 mosM) and suspended in an isotonic buffer. The vesicles are initially dense and phase dark (i). Over time (˜hours), the vesicles become phase light, lose their encapsulant, and rise to the top of the chamber (ii). Subsequently, after longer times (˜days), the vesicles exhibit altered morphology, and finally disintegrate (iii). FIG. 16B shows histograms of “loaded” and “empty” vesicles as they evolve dramatically over the time course of the experiment. At initial times, the distribution is dominated by encapsulant “loaded” carriers (˜90%), whereas after 4 days, dominant fractions (˜80%) of the visible vesicles appear “empty.” Scale bars are 5 μm.

FIGS. 17A and 17B depict phase contrast and fluorescent imaged kinetics of release from giant OL1/OB18 (25:75 mol %) vesicles loaded with a molecular weight series of dextrans in sucrose. FIG. 17A shows in several images that sucrose and the fluorescein-5-isothiocyanate (FITC)-dextran (4.4 kDa) are increasingly released over the 3-day duration of the experiment; but that the large dextran (160 kDa) showed no release. This provides an upper limit to a finite pore size in the membrane. Scale bars are 5 μm. FIG. 17B graphically shows that the indicated release time constants are determined from kinetics.

FIGS. 18A-18C graphically depicts blend-controlled release kinetics of a small encapsulant from various polymer vesicle formulations. FIG. 18A shows that pure OB18 vesicles (0% OL1) porate minimally over time, but poration probability increases as a function of the mole percent of OL1 blended with OB18. The solid lines for 10%, 25%, and 50% blends are fits to A[1 exp (t/τ)] with the indicated release times, t=τᵣₑₗₑₐₛₑ; the dashed line represents the extrapolated kinetics for 100% OL1 vesicles. FIG. 18B shows plotting release kinetics (1/τ) versus mole percent of OL1 blended into the membranes, a first-order rate dependence. FIG. 18C shows release kinetics from 25 mol % blends monitored with various bulk dilutions into PBS. Subsequent, pore induction and deviations in the encapsulant release times are within 15%, making them independent of dilution and exterior factors. In each experiment, the vesicles are suspended in buffered PBS (300 mosM) and incubated in a closed chamber at 25° C.

FIGS. 19A and 19B graphically depict a summary of encapsulant release kinetics from copolymer vesicles as dictated by both chain chemistry and PEG volume fraction (f_{EO}). FIG. 19A shows that OL copolymers of several thousand g/mol can integrate at 25 mol % into stable vesicles of inert OB18 as long as f_{EO}≦0.73 (black-filled star). For pure vesicles of such degradable copolymers (i.e., 100%), release is much faster and requires f_{EO}≦0.42 (open white star). FIG. 19B shows that a OCL copolymer of similar molecular weight as OL1 and OL2, degrades more slowly when accounted for the f_{EO} effect. This delay due to polyester chain chemistry reflects retarded PCL degradation kinetics. A characteristic release line through the result for the 25% OCL blend intersects the 25% OL line at f_{EO}=0.73, where f_{EO} dominates any major difference in degradation chemistry. Likewise, release from pure OCL vesicles can be predicted by postulating slower proportionate degradation, but at a common microphase stability limit of f_{EO}=0.42. Comparing the two OCL1 lines to OCL2 data points reveals the effect of molecular weight or lack thereof since OCL2 is about four-fold bigger than OCL1 or the two OL block copolymers.

FIGS. 20A-20D depict the nuclear delivery of doxorubicin (DOX) via exemplified degradable polymersomes in MDA-MB231 breast cancer epithelial cells. FIG. 20A shows the effectiveness of dual labeling of the polymersome carrier allowing a visual confirmation of “loaded” drug (DOX) (in black and white, the fluorescent encapsulant causes the polymersome to appears as bright white), as compared with “empty” vesicles (appearing in light gray with white fluorescence only at the border). FIG. 20B shows MDA-MB231 cells incubated with FITC-labeled, DOX-loaded degradable polymersomes. Overlays of bright field and fluorescent images are shown demonstrating nuclear localization of DOX (FIG. 20C) and perinuclear localization of the associated polymersomes (FIG. 20D).

FIG. 21 graphically displays the effects of a cytotoxicity assay of the MDA-MB231 cells treated with DOX-loaded degradable polymersomes (OL2/OB18 blended at 25:75 mol % ratio), demonstrating the effective delivery of the encapsulant from the polymersome carrier into the MDA-MB231 cells of FIG. 20.

FIG. 22 graphically displays the effects (by MTT assay) of delivering taxol-loaded hydrolytically degradable polymersomes (OL2/OB18 blended at 25:75 mol % ratio) in human cells at early time points, showing a controlled time released cytotoxic effect of the accumulated, released hydrophobic FITC labeled drug. Cell proliferation was inhibited with taxol loaded degradable polymersomes as shown at time points 1, 12, and 24 hours. Fluorescence microscopy images (not shown) of taxol-loaded vesicles incubated with cells for either 1 or 4 hours demonstrated rapid internalization and perinuclear localization of the drug-loaded vesicles.

### DETAILED DESCRIPTION OF PREFERRED EMBODIMENTS OF THE INVENTION

The present invention provides methods for the controlled release of one or more active agents from stable vesicles, comprising semi-permeable, thin-walled encapsulating membranes, tens of nanometers to tens of microns in diameter, made by self-assembly in various aqueous solutions of purely synthetic, amphiphilic molecules having an average molecular weight of many kilograms per mole. Such molecules are referred to as “super-amphiphiles” because of their large molecular weight in comparison to other amphiphiles, such as the phospholipids and cholesterol of eukaryotic cell membranes.

The relevant class of super-amphiphilic molecules is represented by, but not limited to, block copolymers, e.g., hydrophilic polyethyleneoxide (EO) linked to hydrophobic polyethylethylene (EE). The synthetic diversity of block copolymers provides the opportunity to make a wide variety of vesicles with material properties that greatly expand what is currently available from the spectrum of naturally occurring phospholipids. For the purposes of this invention, although technically distinct and distinguished on the basis of molecular weight, the terms “super-amphiphile” and “amphiphile” are used interchangeably, for example, to refer to the block copolymers of the present invention.

In a preferred embodiment, the invention further provides for the preparation of vesicles harboring mixtures of super-amphiphiles and smaller amphiphiles, such as phospholipids up to at least 20% mole fraction. The latter have been shown to be capable of integrating into stable vesicles of super-amphiphiles.

“Vesicles,” as the term is used in the present invention, are essentially semi-permeable bags of aqueous solution as surrounded (without edges) by a self-assembled, stable membrane composed predominantly, by mass, of either amphiphiles or super-amphiphiles. Thus, a biological cell would, in general, represent a naturally occurring vesicle. Smaller vesicles are also found within biological cells, and many of the structures within a cell are vesicular. The membrane of an internal vesicle serves the same purpose as the plasma membrane, i.e., to maintain a difference in composition and an osmotic balance between the interior of the vesicle and the exterior. Many additional functions of cell membranes, such as in providing a two-dimensional scaffold for energy conversion can be added to compartmentalization roles. For an intracellular vesicle, the environment outside the vesicle is the cytoplasm.

The “cell membrane” or “plasma membrane” is a complex, contiguous, self-assembled, complex fluid structure comprised of amphiphilic lipids in a bilayer with associated proteins and which defines the boundary of every cell. It is also referred to as a “biomembrane.” “Phospholipids” comprise lipid substances, which occur in cellular membranes and contain esters of phosphoric acid, such as sphingomyelins, and include phosphatides, phospholipins and phospholipoids.

Synthetic amphiphiles having molecular weights less than a few kilodaltons, like natural amphiphiles, are pervasive as self-assembled, encapsulating membranes in water-based systems. These include complex fluids, soaps, lubricants, microemulsions consisting of oil droplets in water, as well as biomedical devices such as vesicles. An “encapsulating membrane,” as the term is used in the present invention, is a vesicle in all respects except for the necessity of aqueous solution. Encapsulating membranes, by definition, compartmentalize by being semi- or selectively permeable to solutes, either contained inside or maintained outside of the spatial volume delimited by the membrane. Thus, a vesicle is a capsule in aqueous solution, which also contains aqueous solution. However, the interior or exterior of the capsule could also be another fluid, such as an oil or a gas. A “capsule,” as the term is used in the present invention, is the encapsulating membrane plus the space enclosed within the membrane.

“Complex fluids” are fluids that are made from molecules that interact and self-associate, conferring novel Theological, optical, or mechanical properties on the fluid itself. Complex fluids are found throughout biological and chemical systems, and include materials such as biological membranes or biomembranes, polymer melts and blends, and liquid crystals. The self-association and ordering of the molecules within the fluid depends on the interaction between component parts of the molecules, relative to their interaction with solvent, if present.

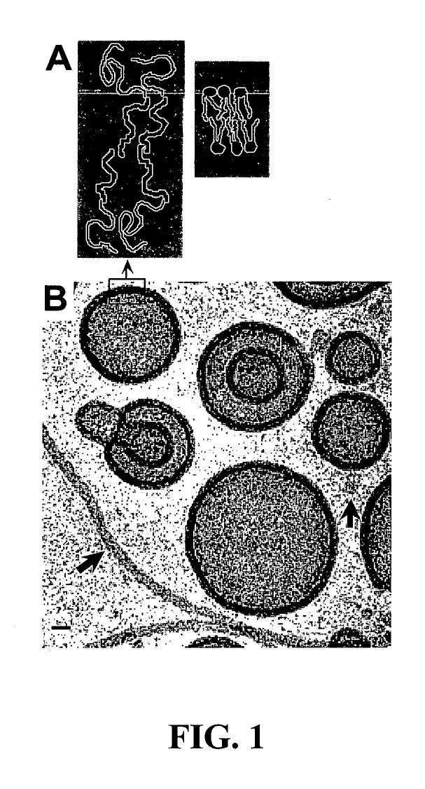

The plasma membrane is a “lipid bilayer” comprising a double layer of phospholipid/diacyl chains, wherein the hydrophobic fatty acid tails of the phospholipids face each other and the hydrophilic polar heads of each layer face outward toward the aqueous solutions (see FIG. 1A). Numerous receptors, steroids, transporters and the like are embedded within the bilayer of a typical cell. Thus, a “lipid vesicle” or “liposome,” is a vesicle surrounded by a membrane comprising one or more phospholipids. Throughout the specification the terms “cell membrane,” “plasma membrane,” “lipid membrane,” and “biomembrane” may be used interchangeably to refer to the same lipid bilayer surrounding a cell or vesicle.

A “membrane”, as the term is used in this invention, is a spatially distinct collection of molecules that defines a 2-dimensional surface in 3-dimensional space, and thus separates one space from another in at least a local sense. Such a membrane must also be semi-permeable to solutes. It must also be sub-microscopic (less than optical wavelengths of around 500 nm) in its thickness (d in FIG. 1A), as resulting from a process of self-assembly. It can have fluid or solid properties, depending on temperature and on the chemistry of the amphiphiles from which it is formed. At some temperatures, the membrane can be fluid (having a measurable viscosity), or it can be solid-like, with an elasticity and bending rigidity. The membrane can store energy through its mechanical deformation, or it can store electrical energy by maintaining a transmembrane potential. Under some conditions, membranes can adhere to each other and coalesce (fuse). Soluble amphiphiles can bind to, and intercalate within a membrane.

A “bilayer membrane” (or simply “bilayer(s)”) for the purposes of this invention is a self assembled membrane of amphiphiles or super-amphiphiles in aqueous solutions.

“Polymersomes” are vesicles, which are assembled from synthetic multi-block polymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self assembly,” a spontaneous, entropy-driven process of preparing a closed semi-permeable membrane.

Because of the perselectivity of the bilayer, materials may be “encapsulated” in the aqueous interior (lumen) or intercalated into the hydrophobic membrane core of the polymersome vesicle of the present invention, forming a “loaded polymersome.” Numerous technologies can be developed from such vesicles, owing to the numerous unique features of the bilayer membrane and the broad availability of super-amphiphiles, such as diblock, triblock, or other multi-block copolymers.

The synthetic polymersome membrane can exchange material with the “bulk,” i.e., the solution surrounding the vesicles. Each component in the bulk has a partition coefficient, meaning it has a certain probability of staying in the bulk, as well as a probability of remaining in the membrane. Conditions can be predetermined so that the partition coefficient of a selected type of molecule will be much higher within a vesicle's membrane, thereby permitting the polymersome to decrease the concentration of a molecule, such as cholesterol, in the bulk. In a preferred embodiment, phospholipid molecules have been shown to incorporate within polymersome membranes by the simple addition of the phospholipid molecules to the bulk. In the alternative, polymersomes can be formed with a selected molecule, such as a hormone, incorporated within the membrane, so that by controlling the partition coefficient, the molecule will be released into the bulk when the polymersome arrives at a destination having a higher partition coefficient.

The polymersomes of the present invention are formed from synthetic, amphiphilic copolymers. An “amphiphilic” substance is one containing both polar (water-soluble) and hydrophobic (water-insoluble) groups. “Polymers” are macromolecules comprising connected monomeric units. The monomeric units may be of a single type (homogeneous), or a variety of types (heterogeneous). The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains). For example, in polyethylene glycol (PEG), which is a polymer of ethylene oxide (EO), the chain lengths which, when covalently attached to a phospholipid, optimize the circulation life of a liposome, is known to be in the approximate range of 34-114 covalently linked monomers (EO₃₄ to EO₁₁₄).

The preferred class of polymer selected to prepare the polymersomes of the present invention is the “block copolymer.” Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent. Thus, an enormous spectrum of block chemistries is theoretically possible, limited only by the acumen of the synthetic chemist.

In the “melt” (pure polymer), a diblock copolymer may form complex structures as dictated by the interaction between the chemical identities in each segment and the molecular weight. The interaction between chemical groups in each block is given by the mixing parameter or Flory interaction parameter, χ, which provides a measure of the energetic cost of placing a monomer of A next to a monomer of B. Generally, the segregation of polymers into different ordered structures in the melt is controlled by the magnitude of χN, where N is proportional to molecular weight. For example, the tendency to form lamellar phases with block copolymers in the melt increases as χN increases above a threshold value of approximately 10.

A linear diblock copolymer of the form A-B can form a variety of different structures. In either pure solution (the melt) or diluted into a solvent, the relative preferences of the A and B blocks for each other, as well as the solvent (if present) will dictate the ordering of the polymer material. In the melt, numerous structural phases have been seen for simple AB diblock copolymers.

To form a stable membrane in water, the absolute minimum requisite molecular weight for an amphiphile must exceed that of methanol HOCH₃, which is undoubtedly the smallest canonical amphiphile, with one end polar (HO—) and the other end hydrophobic (—CH₃). Formation of a stable lamellar phase more precisely requires an amphiphile with a hydrophilic group whose projected area, when viewed along the membrane's normal, is approximately equal to the volume divided by the maximum dimension of the hydrophobic portion of the amphiphile (Israelachvili, in Intermolecular and Surface Forces, 2^{nd} ed., Pt3 (Academic Press, New York) 1995).

The most common lamellae-forming amphiphiles also have a hydrophilic volume fraction between 20 and 50%. Such molecules form, in aqueous solutions, bilayer membranes with hydrophobic cores never more than a few nanometers in thickness. The present invention relates to all super-amphiphilic molecules which have hydrophilic block fractions within the range of 20-50% by volume and which can achieve a capsular state. The ability of amphiphilic and super-amphiphilic molecules to self-assemble can be largely assessed, without undue experimentation, by suspending the synthetic super-amphiphile in aqueous solution and looking for lamellar and vesicular structures as judged by simple observation under any basic optical microscope or through the scattering of light.

For typical phospholipids with two acyl chains, temperature can affect the stability of the thin lamellar structures, in part, by determining the volume of the hydrophobic portion. In addition, the strength of the hydrophobic interaction, which drives self-assembly and is required to maintain membrane stability, is generally recognized as rapidly decreasing for temperatures above approximately 50° C. Such vesicles generally are not able to retain their contents for any significant length of time under conditions of boiling water.

Upper limits on the molecular weight of synthetic amphiphiles which form single component, encapsulating membranes clearly exceed the many kilodalton range, as concluded from the work of Discher et al., (1999), which contributes foundationally to the present invention, and is herein incorporated by reference.

Block copolymers with molecular weights ranging from about 2 to 10 kilograms per mole can be synthesized and made into vesicles when the hydrophobic volume fraction is between about 20% and 50%. Diblocks containing polybutadiene are prepared, for example, from the polymerization of butadiene in cyclohexane at 40° C. using sec-butyllithium as the initiator. Microstructure can be adjusted through the use of various polar modifiers. For example, pure cyclohexane yields 93% 1.4 and 7% 1.2 addition, while the addition of THF (50 parts per Li) leads to 90% 1.2 repeat units. The reaction may be terminated with, for example, ethyleneoxide, which does not propagate with a lithium counterion and HCl, leading to a monofunctional alcohol. This PB—OH intermediate, when hydrogenated over a palladium (Pd) support catalyst, produces PEE-OH. Reduction of this species with potassium naphthalide, followed by the subsequent addition of a measured quantity of ethylene oxide, results in the PEO-PEE diblock copolymer. Many variations on this method, as well as alternative methods of synthesis of diblock copolymers are known in the art; however, this particular preferred method is provided by example, and one of ordinary skill in the art would be able to prepare any selected diblock copolymer.

For example, if PB-PEO diblock copolymers were selected, the synthesis of PB-PEO differs from the previous scheme by a single step, as would be understood by the practitioner. The step by which PB-OH is hydrogenated over palladium to form PEO-OH is omitted. Instead, the PB-OH intermediate is prepared, then it is reduced, for example, using potassium naphthalide, and converted to PB-PEO by the subsequent addition of ethylene oxide.

In yet another example, triblock copolymers having a PEO end group can also form polymersomes using similar techniques. Various combinations are possible comprising, e.g., polyethylene, polyethylethylene, polystyrene, polybutadiene, and the like. For example, a polystyrene (PS)-PB-PEO polymer can be prepared by the sequential addition of styrene and butadiene in cyclohexane with hydroxyl functionalization, re-initiation and polymerization. PB-PEE-PEO results from the two-step polymerization of butadiene, first in cyclohexane, then in the presence of THF, hydrolyl functionalization, selective catalytic hydrogenation of the 1.2 PB units, and the addition of the PEO block.

A plethora of molecular variables can be altered with these illustrative polymers, hence a wide variety of material properties are available for the preparation of the polymersomes. ABC triblocks can range from molecular weights of 3,000 to at least 30,000 g/mol. Hydrophilic compositions should range from 20-50% in volume fraction, which will favor vesicle formation. The molecular weights must be high enough to ensure hydrophobic block segregation to the membrane core. The Flory interaction parameter between water and the chosen hydrophobic block should be high enough to ensure said segregation. Symmetry can range from symmetric ABC triblock copolymers (where A and C are of the same molecular weight) to highly asymmetric triblock copolymers (where, for example, the C block is small, and the A and B blocks are of equal length).

TABLE 1 lists some of the synthetic super-amphiphiles of many kilograms per mole in molecular weight, which are capable of self-assembling into semi-permeable vesicles in aqueous solution. The panel of preferred PEO-PEE block copolymers ranges in molecular weight from 1400 to 8700, with hydrophilic volume fraction, f_{EO}, ranging from 20% to 50%. The polydispersity indices for the resulting polymers do not exceed 1.2, confirming a narrow polydispersity.

TABLE 1

| | Molecular Weight | Vol. fraction EO |
|---|---|---|
| Super-Amphiphile* | (g/mol)** | (±1%)^{‡} |
| EO₄₀-EE₃₇ | 3900 | 39% |
| EO₄₃-EE₃₅ | 3900 | 42% |
| EO₄₉-EE₃₇ | 4300 | 44% |
| EO₂₆-PB₄₆ | 3600 | 28% |
| EO₃₁-PB₄₆ | 3800 | 31% |
| EO₄₂-PB₄₆ | 5300 | 37% |
| EO₃₃-S₁₀-I₂₂^{‡‡} | 3900 | 33% |
| EO₄₈-EE₇₅-EO₄₈ | 8400 | 44% |

*EO = ethyleneoxide, EE = ethylethylene, B = butadiene, S = styrene, I = isoprene
**Molecular Weight denotes number-average molecular weight (Mn) ± 50 g/mol
^{‡}Volume fractions determined by NMR.
^{‡‡}EO-S-I has number-average molecular weight for the respective blocks of 1440, 1008, 1470 g/mol.

TABLE 1 is intended only to be representative of the synthetic super-amphiphiles suitable for use in the present invention. It is not intended to be limiting. The table can be effectively used to select which block copolymers will form lamellar phases and vesicles. One of ordinary skill in the art will readily recognize many other suitable block copolymers that can be used in the preparation of polymersomes based on the teachings of the present invention.

In a preferred embodiment of the present invention, polymersomes comprise the selected polymer polyethyleneoxide-polyethylethylene (EO₄₀-EE₃₇), also designated OE-7, and having a chain structure t-butyl-[CH₂—CH(C₂H₅)]₃₇-[CH₂—CH₂—O]₄₀—H. The molecule's average molecular weight is about five to ten times greater than that of typical phospholipids in natural membranes. The resulting polymersome membrane is found to be at least 10 times less permeable to water than common phospholipid bilayers.

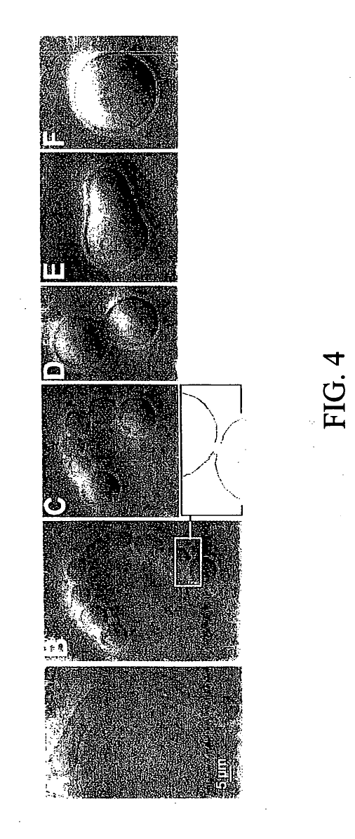

A vesicle suspended in water which encapsulates impermeable solutes and which has a non-zero membrane permeability to water can be osmotically forced to change its shape. Shape transformations of vesicle capsules, the simple red blood cell included, have generally been correlated with energy costs or constraints imposed by vesicle area, the number of membrane molecules making up the vesicle area, the volume enclosed by the vesicle, and the curvature elasticity of the membrane (see, e.g., Deuling et al., J. Phys. 37:1335 (1976); Svetina et al., Eur. Biophys. 17:101 (1989); Seifert et al., Phys. Rev. A 44:1182 (1991)). Theoretical and experimental efforts on fluid lipid bilayers (e.g., Seifert and Lipowsky, in Handbook of Biological Physics, chap. 8; Dobereiner et al., Phys. Rev. E 55:4458 (1997)) have separated the elasticity in bending between a local, K_{b}-scaled curvature energy term that includes a spontaneous curvature, c₀, and a more non-local, area-difference-elasticity term predicated on monolayer unconnectedness in spherical-topology vesicles. To oppose any relaxation of leaflet area difference, a lack of lipid transfer or “flip-flop” between layers must be postulated. Only with such a non-local area difference term can a vesicle maintain in apparent equilibrium the type of multi-sphere and budded morphologies observable in both lipid systems (Chaieb et al., Phys. Rev. E 58:7733 (1998)) and in the osmotically deflated polymersomes shown in FIG. 4. Because worm-like and spherical micelles are also in evidence (FIG. 1B), however, a non-zero cₒ also appears likely. Heterogeneity in the morphology of polymersomes, both small (FIG. 1B) and large vesicles (FIG. 4), denotes, however, an important contribution from monolayer area difference, a process-dependent feature that arises upon vesicle closure.

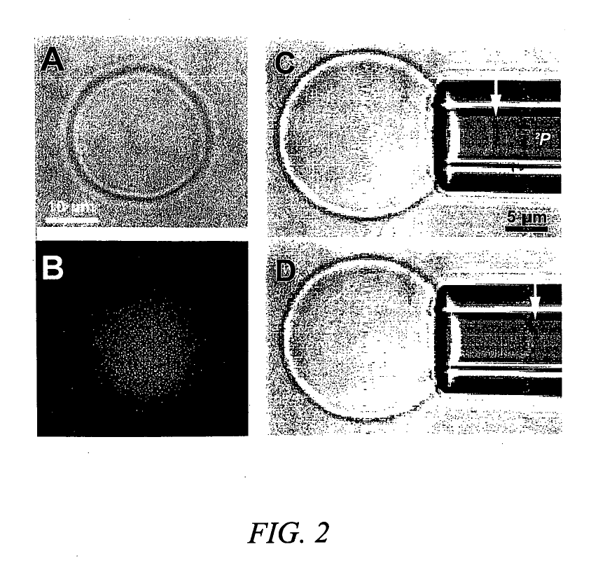

The tool that has been used to measure many of the material properties of bilayer vesicles is “micropipette aspiration” as applied in FIG. 2. In micropipette aspiration, the rheology and material properties of micron-sized objects are measured using glass pipettes. Small, micron diameter pipettes are used to pick up, deform and manipulate micron-sized objects, such as giant lipid vesicles. The aspiration pressure is controlled by manometers, in which the hydrostatic pressure in a reservoir connected to the micropipette is varied in relation to a fixed reference. Pressure may be varied with a resolution of microns of H₂O (or 10⁻⁶ atm).

A deformable object is aspirated using a pressure driving force (or suction pressure), ΔP, and the object is drawn within the pipette to a projection length L_{P}. For a liquid, the tension in the membrane, τ, can be obtained from the Law of Laplace in terms of the pressure driving force, the pipette inner radius, R_{P}, the vesicular outer diameter, R_{S}, and the length of the projection. This technique has been used to measure the moduli of deformation and strength of lipid vesicle membranes, such as the bending modulus (K_{b}), the area expansion modulus (Kₐ), the critical areal strain to the point of failure (α_{c}) and the toughness (E_{c} or T_{f}) (the energy stored in the vesicle prior to failure) (see, e.g., Evans et al., J. Phys. Chem. 91:4219 (1987); Needham et al., Biophys. J. 58:997 (1990)). The bending modulus is measured by exerting small tensions on the membrane, to smooth out thermally-driven surface undulations. At larger tensions, beyond a crossover tension at which the undulations of the membrane have been smoothed, the tension acts to stretch the membrane in-plane against the cohesive hydrophobic forces holding the membrane together. The area expansion modulus is the unit tension required for a unit increase in strain. The critical area strain is obtained by stressing the membrane to the point of cohesive failure. Thus, micropipette aspiration is a powerful tool for exploring the interfacial and material properties of the polymersomes of the present invention.

TABLE 2 demonstrates that the membrane mechanical properties of several preferred polymer vesicles are independent of the different methods of assembly in aqueous media Kₐ falls within the broad range of lipid membrane measurements. In contrast, the giant polymersomes of the present invention prove to be almost an order of magnitude tougher and sustain far greater areal strain under tension before rupture than any naturally occurring or synthetic vesicle known in the art. Membranes formed from the preferred super-amphiphilic diblocks of either polyethyleneoxide-polyethylethylene or polyethyleneoxide-polybutadiene have also been shown to be thicker than lipid membranes, providing a physical basis for understanding the enhanced toughness, as well as the reduced permeability.

TABLE 2

| Super- | Method of | | | |
|---|---|---|---|---|
| Amphiphile | Formation | Kₐ (mN/m)* | α_{c} = (ΔA/Ao)** | d: thickness*** |
| EO₄₀-EE₃₇ | Film Rehydration | 115 ± 27 [20 vesicles] | 0.20 ± 0.07 [5 vesicles] | 8 ± 1 nm |
| | Electroformation | 120 ± 20 [21 vesicles] | 0.19 ± 0.02 [6 vesicles] | |
| EO₂₆-B₄₆ | Film Rehydration | 80 ± 34 [5 vesicles] | | 9 ± 1 nm |
| | Bulk Rehydration | 94 ± 10 [4 vesicles] | | |
| EO₅₀-B₅₄ | Film Rehydration | 82 ± 23 [9 vesicles] | 0.30 [2 vesicles] | |

*Kₐ is the elastic modulus for area expansion.
**α_{c} is the critical area strain at which an initially unstressed membrane will rupture.
***The hydrophobic core thickness, d, is determined by electron microscopy.

Preferred assemblies of the present invention can withstand exceptionally severe environmental conditions of temperature and exposure to solvent. TABLE 3 indicates the result of suspending vesicles of EO₄₀-EE₃₇ in a sterilizing aqueous solution of ethanol in phosphate buffered saline (PBS) for at least 15 minutes. Many phospholipid vesicles would be unstable under such solvent conditions.

TABLE 3

| | 25% EtOH in PBS | PBS |
|---|---|---|
| Vesicle per ml* | 7.2 × 10⁴ | 9.0 × 10⁴ |
| Vesicle diameter (μm) | 9.7 ± 5.4 | 8.6 ± 4.1 |

*5 μl of vesicles in 247 mOsm sucrose were added to 200 μl of 25% EtOH/PBS or PBS.

The methods and examples that follow make use of and extend the above characterization methods and concepts.

A. Preparation of Polymersomes

In the preferred embodiments of the present invention, the polymersomes are comprised of a subset class of block copolymers—the “amphiphilic block copolymers,” meaning that in a diblock copolymer, region A is hydrophilic and region B is hydrophobic. Like phospholipid amphiphiles, block copolymer amphiphiles self-assemble into lamellar phases at certain compositions and temperatures and can form closed bilayer structures capable of encapsulating aqueous materials. Vesicles from block-copolymer amphiphiles have the additional advantage of being made from synthetic molecules, permitting one of ordinary skill to apply known synthetic methods to greatly expand the types of vesicles and the material properties that are possible based upon the presently disclosed and exemplified applications.

The diblock copolymers used to form the super-amphiphile vesicles of the invention may be synthesized by any method known to one of ordinary skill in the art for synthesizing copolymers. Such methods are taught, for example, by Hajduk et al., 1998; Hillmyer and Bates, Macromolecules 29, 6994-7002 (1996); and Hillmyer et al., Science 271:976 (1996)), although the practitioner need not be so limited. Nevertheless, use of the Bates method results in very low polydispersity indices for the synthesized polymer (not exceeding 1.2), and make the methods particularly suited for use in the present invention, at least from the standpoint of homogeneity. Indeed, the demonstrated ability to make stable vesicles from PEO-PEE with up to at least 20% mole fraction of phospholipid strongly indicates that polydispersity need not be limiting in the formation of stable vesicles.

Vesicles can be prepared by any method known to one of ordinary skill in the art. However, the preferred method of preparation is film rehydration, which has yielded vesicles for all copolymers that have been found to be capable of forming vesicles. Other methods can be used as described below, but they do not guarantee vesicle formation for all “vesicle-forming” amphiphiles.

(1) Film Rehydration

In the film rehydration method, in general, pure amphiphiles are dissolved in any suitable solvent that can be completely evaporated without distracting the amphiphile, at concentrations preferably ranging from 0.1 to 50 mg/ml, more preferably from 1 to 10 mg/ml, most preferably yielding 1 μmol/ml solution. The preferred solvent for this purpose in the present invention is chloroform. When amphiphile mixtures are used, each component of the mixture must be dissolved separately and mixed in a measured aliquot of the solvent to obtain a solution comprising the desired ratio of components. The resulting solution is placed into a glass vial, and the solvent is evaporated to yield a thin film, having a preferable density of approximately 0.01 μmol/cm².

When chloroform is used as the solvent, the solution is evaporated under nitrogen gas and under applied vacuum for three hours or longer, until evaporation is completed. After complete evaporation of the solvent, an aqueous solution comprising the “to be encapsulated” material is added to the glass vial, yielding a preferred 0.1% (w/w) solution. Vesicles form spontaneously at room temperature in a time dependent manner ranging from several hours to several days, depending on the selected amphiphile and the aqueous solvent and the ratio between them. Temperature may be used as a control variable in this process of formation. The yield of vesicles can be optimized without undue experimentation by the selection of aqueous components and by tuning the experimental conditions, such as concentration and temperature.

(2) Bulk Rehydration

In the alternative, the pure amphiphile can be mixed with an aqueous solution to a preferred concentration of 0.01-1% (w/w), most preferably 0.1% (w/w), then dissolved into small aggregates (with dimensions of several microns) by mixing. When the aggregates are then incubated without any perturbance for several hours to several days, depending on the amphiphile, aqueous solvent and temperature, vesicles form spontaneously on the aggregate surface, from which they can be dissociated by gentle mixing or shaking.

(3) Electroformation

Polymersomes are more preferably made by electroformation, by using the adapted methods of Angelova et al., Prog. Coll. Polym. Sci. 89:127 (1992), which have been previously used by Hammer as reported by Longo et al., Biophys. J. 73:1430 (1997) (both are herein incorporated by reference), although the preparation need not be so limited. Briefly, by example, 20 μl of the amphiphile solution (in chloroform or other solvent made to preferable concentration 1 μmol/ml) is deposited as a film on two 1 mm-diameter adjacent platinum wire electrodes held in a Teflon frame (5 mm separation of the electrodes). The solvent is then evaporated under nitrogen, followed by vacuum drying for 3 to 48 hours. The Teflon frame and coated electrodes are then assembled into a chamber, which is then sealed with coverslips. Preferably, the temperature and humidity of the chamber are controlled. The chamber is subsequently filled with a degassed aqueous solution, e.g., glucose or sucrose, preferably about 0.1 to 0.25 M or with a protein solution containing, for example, a globin.

To begin generating polymersomes from the film, an alternating electric field is applied to the electrodes (e.g., 10 Hz, 10 V) while the chamber is mounted and viewed on the stage of an inverted microscope. Giant vesicles attached to the film-coated electrode are visible after 1 to 60 min. The vesicles can be dissociated from the electrodes by lowering the frequency to about 3 to 5 Hz for at least 15 min, and by removing the solution from the chamber into a syringe.

In spite of several techniques used, it was found in practicing the present invention, that for each of the particular amphiphiles studied, the method selected for vesicle formation did not alter the mechanical properties of the resulting vesicles (TABLE 2).

(4) Fragmentation

The size of giant polymersome can be decreased to any average vesicle size as desired for a given application by filtration through polycarbonate filter (Osmonics, Livermore, Calif.). As an example, 5.5±3.0 μm vesicles were filtered through a 1.0 micron polycarbonate filter. The size of the vesicles decreased to 2.4±0.36 μm.

B. Characterization of Polymersomes

The structure of an exemplified polymersome vesicle can be characterized by the following generalized method. In a preferred embodiment, 1% (w/w) of the amphiphile is solubilized in aqueous solution, and the vesicles self-assemble during the solubilization process. Thin films (ca. 100 nm) of the vesicular solution suspended within the pores of a microperforated grid are prepared in an isolated chamber with controlled temperature and humidity (Lin et al., Langmuir, 8:2200 1992). The sample assembly is then rapidly vitrified with liquid ethane at its melting temperature (˜90 K), and then kept under liquid nitrogen until loaded onto a cryogenic sample holder (Gatan 626) (Lin et al., (1992)).

The morphologies of the polymersomes may be visualized by cryo-transmission electron microscopy (cryo-TEM or CTEM), by transmission electron microscopy (TEM), such as on a Phillips EM410 transmission electron microscope operating at an acceleration voltage of 80-100 kV, by inverted stage microscopy, or by any other means known in the art for visualizing vesicles. Cryo-TEM images revealed, at 1 nm resolution, the mean lamellar thickness of the hydrophobic core, which was ˜8 to 9 nm for both the EO₄₀-EE₃₇ and EO₂₆-PD₄₆ membranes as listed in TABLE 2.

Small angle X-ray and neutron scattering (SAXS and SANS) analyses are well suited for quantifying the thickness of the membrane core (Won et al., 1999) or any internal structure. SAXS and SANS can provide precise characterization of the membrane dimensions, including the conformational characteristics of the PEO corona that stabilizes the polymersome in an aqueous solution. Neutron contrast is created by dispersing the vesicles in mixtures of H₂O and D₂O, thereby exposing the concentration of water as a function of distance from the hydrophobic core.

Size distribution can be determined directly by microscopic observation (light and/or electron microscopy), by dynamic light scattering, or by other known methods.

Polymersome vesicles can range in size from tens of nanometers to hundreds of microns in diameter. According to accepted terminology developed for lipid vesicles, small vesicles can be as small as about 1 nm in diameter to over 100 nm in diameter, although they typically have diameters in the tens of nanometers. Large vesicles range from 100 to 500 run in diameter. Both small and large vesicles are best perceived as such by light scattering and electron microscopy. Giant vesicles are generally greater than 0.5 to 1 μm in diameter, and can generally be perceived as vesicles by optical microscopy.

Small vesicles can be as small as 1 nm in diameter to over 100 nm in diameter, although they typically have diameters in the tens of nanometers. Large vesicles range from 100 to 1000 nm in diameter, preferably from 500 to 1000 run. Giant vesicles are generally greater than 1 μm in diameter. The preferred polymersome vesicles range of 20 nm to 100 μm, preferably from 1 μm to 75 μm, and more preferably from 1 μm to 50 μm.

The disclosed methods of preparation of the polymersomes are particularly preferred because the vesicles are prepared without the use of co-solvent. Any organic solvent used during the disclosed synthesis or film fabrication method has been completely removed before the actual vesicle formation. Therefore, the polymersomes of the present invention are free of organic solvents, distinguishing the vesicles from those of the prior art and making them uniquely suited for bio-applications.

The methods of analysis applied in a preferred embodiment of the invention provide a clear basis for applications of mass retention, delivery, and extraction, which may require membrane biocompatibility, and which may or may not take advantage of the novel thermal, mechanical, or chemical properties of the membranes. By “biocompatible” is meant a substance or composition which can be introduced into an animal, particularly into a human, without significant adverse effect. For example, when a material, substance or composition of matter is brought into a contact with a viable white blood cell, if the material, substance or composition of matter is toxic, reactive or biologically incompatible, the cells will perceive the material as foreign, harmful or immunogenic, causing activation of the immune response, and resulting in immediate, visible morphological changes in the cell. A “significant” adverse effect would be one which is considered sufficiently deleterious as to preclude introducing a substance into the patient.

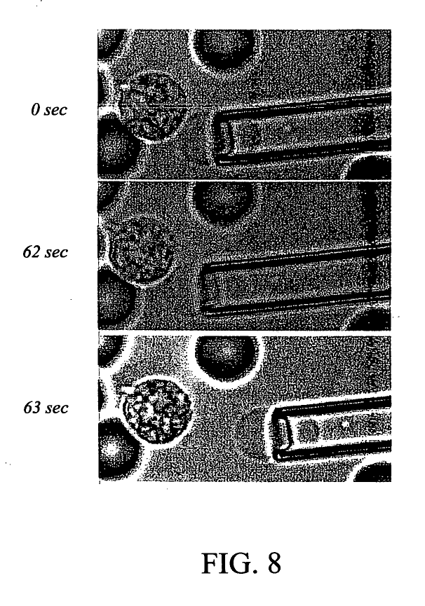

To confirm one level of biocompatibility of the polymersomes, preliminary evaluations were performed by bringing the polymersomes into contact with white blood cells, such as granulocytes, as seen in FIG. 8A. Even after prolonged contact (over one minute) with the polymersomes, the white cells remained intact and unchanged (FIGS. 8B and 8C). No adhesion was observed, and the polymersomes caused no activation of the white blood cells, thus demonstrating the biocompatibility of the polymersomes.

If there were adhesion between vesicles and blood cells, micropipette aspiration could also be used to measure the inter-lamellar adhesion energy. If two vesicles or a cell and vesicle are manipulated into contact and adherent, then the inter-lamellar adhesion energy density 7 is determined from Young's equation, γ=τ(1−cos θ), where θ is the measurable contact angle between the two surfaces, τ is the tension required to peel the membranes apart. In the case of adhesion being strong enough to induce membrane cohesion, aspiration can again be used to directly observe the resulting coalescence of two vesicles (fusion), as well as the adsorption and intercalation of soluble objects (such as, surfactants or micelles) into the membrane.

C. Encapsulation into Polymersomes

An enormously wide range of encapsulants (or active agents), either hydrophilic or hydrophobic, can be encapsulated within a polymersome vesicle. In fact, to date no molecule has been found that cannot be encapsulated. Hydrophobic agents integrate into the membrane; whereas hydrophilic agents are contained within the vesicle's aqueous lumen of the vesicle.

Among the exemplary molecules that have been encapsulated are: proteins and proteinaceous compositions, e.g., myoglobin, hemoglobin and albumin, sugars and other representative carriers for drugs, therapeutics and other biomaterials, e.g., 10 kDa dextran, sucrose, and phosphate buffered saline, as well as marker preparations. Encapsulation applications range, without limitation from, e.g., drug delivery (aqueously soluble drugs), to optical detectors (fluorescent dyes), to the storage of oxygen (hemoglobin).

A variety of fluorescent dyes of the type that can be incorporated within the polymersomes could include small molecular weight fluorophores, such as fluorescein-5-isothiocyanate (FITC), and fluorophores attached to dextrans of a laddered sequence of molecular weights. Imaging of the fluorescent core can be accomplished by standard fluorescent videomicroscopy. Permeability of the polymersome to the fluorophore can be measured by manipulating the fluorescently-filled vesicle with aspiration, and monitoring the retention of fluorescence against a measure of time.

Phosphate buffered saline (PBS; 10 mM phosphate buffer, 2.7 mM KCl, and 137 M NaCl) and other electrolytes or salts, such as, but not limited to, KF or KI can be added during the vesicle preparation and be easily encapsulated by rehydration. The electroformation method is not very efficient in the presence of electrolytes.

TABLE 4 sets forth an exemplary list of compositions that have been successfully loaded into and subsequently delivered from polymersomes. While the listed compositions are not intended to be limiting, both hydrophilic and hydrophobic compositions have been delivered by controlled release from the degradable polymersomes of Example 5. In fact, since the loaded encapsulants reside in different parts of the polymersome, more than one encapsulant can be loaded, i.e., both hydrophilic and hydrophobic compositions.

TABLE 4
Compounds loaded in degradable polymersomes.

| | | | Hydrophobic: |
|---|---|---|---|
| Class of Loaded | | Hydrophilic: in | integrated |
| compound | Loaded compound | vesicle lumen | in membrane |
| Cytotoxic Drug | Taxol | | X |
| fluorescent dyes | PKH membrane dye | | X |
| Cytotoxic Drug-Dye | Fluorescein-Taxol | | X |
| Fluorescent-dye | Degradable and Inert | | X |
| modified Copolymers | Amphiphilic Copolymers | | |
| Cytotoxic Drug | Doxorubicin | X | |
| Fluorescent-dye modified | Fluorescent dextrans from | X | |
| Polymers | ˜1 kD to 200 kD | | |
| Protein | Catalase | X | |
| Nucleic Acids | Oligonucleotides & | X | |
| | Fluorescent-Oligos. | | |
| Carbohydrates | Sucrose, dextrans | X | |

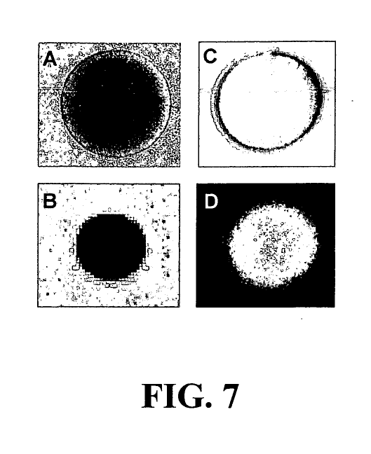

FIG. 7 demonstrates the encapsulation of globular proteins by film rehydration. As shown, EO₄₀-EE₃₇ vesicles were electroformed with 10 g/L myoglobin dissolved in 289 mOsm sucrose solution (FIG. 7A), and with 10 g/L hemoglobin dissolved in 280 mOsm PBS/sucrose solution (FIG. 7B). FIGS. 7C and 7D show a polymer vesicle containing fluorescein-tagged bovine serum albumin (BSA) encapsulated at 0.5 g/l.

D. Cross-Linking of the Polymersomes

In a preferred embodiment, the invention provides reactive amphiphiles that can be covalently cross-linked together, over a many micron-squared surface, while maintaining the semi-permeability of the membrane. Cross-linked polymersomes are particularly useful in applications requiring stability of the vesicle membranes and durable retention of the encapsulated materials. By cross-linked is meant covalently interconnected; i.e., completely cross-linked vesicle have all the membrane components covalently interconnected into a giant single molecule; cross-linked vesicles have interconnected components throughout their entire surface; and partly cross-linked vesicles contain patches of the interconnected components.

Cross-linking of the amphiphiles can be achieved using double bond-containing blocks, such as polybutadiene, which can be readily coupled by standard cross-linking reactions. In a preferred embodiment of the present invention, the vesicles are cross-linked by free radicals generated with combination of an initiator, such as K₂S₂O₈, and a redox coupler, such as Na₂S₂O₅/FeSO₄.7H₂O (Won et al., 1999). Although any suitable pairing of an initiator and a redox coupler may be selected by one of ordinary skill in the art to cause the cross-linking reaction, the suggested compounds have been found to be particularly suited to effect the cross-linking of the exemplified amphiphiles of the present invention. In the preferred and exemplified embodiment, the osmolarity of the cross-linking reagents is adjusted to match the osmolarity of the encapsulated material, and the components are mixed in the following order and volume ratios relative to sample: K₂S₂O₈:Na₂S₂O₅:FeSO₄=1:0.5:0.02. Due to instabilities of the sulfates, K₂S₂O₈ and Na₂S₂O₅ must be prepared within a few days of performing the reaction and FeSO₄ within several minutes of its use to ensure efficient cross-linking of the amphiphiles.

Of course, the cross-linking mechanism need not to be limited to redox reaction methods, such as the one disclosed above. Cross-linking can be carried out by a variety of alternative and known techniques, including but not limited to, ⁶⁰Co γ-irradiation (Hentze et al., Macromolecules 32: 5803-5809 (1999)), or by visible or UV light irradiation with an incorporated sensitizer, such as 3,3,3′,3′-tetramethyldiocta-decyl indocarbocyanine (DiI(C₁₈)). (DiI(C₁₈) is an amphiphilic sensitizing dye which can generate oxygen free radicals when irradiated with green or UV light (Mueller et al., Polymer Preprints (ACS) 40(2):205 (1999)). It has already been established that this particular dye, as well as other dyes, can be incorporated into the polymersome membrane during vesicle preparation, or even after vesicle formation, in relatively large amounts as observed by fluorescent microscopy.

E. Permeability of the Polymersome Membrane, and Transport of Encapsulated Material

(1) Water Permeability

Polymersomes, as exemplified by EO₄₀-EE₃₇, can be substantially less permeable to water than phospholipid membranes, which suggests many beneficial applications for the polymersomes. To measure the permeability of a polymersome to water, observations were made of the time course for vesicle swelling in response to a step change in external medium osmolarity. Briefly, vesicles were prepared in the preferred and exemplified embodiment in 100 mOsm sucrose solution to establish an initial, internal osmolarity, after which they were suspended in an open-edge chamber formed between cover slips, and containing 100 mOsm glucose. A single vesicle was aspirated into a micropipette with a suction pressure sufficient to smooth membrane fluctuations. The pressure was then lowered to a small holding pressure. Using a second, transfer pipette, the vesicle was moved to a second chamber containing 120 mOsm glucose.

When water flows out of the vesicle due to the osmotic gradient between inside and outside of the vesicle, the result is an increased projection length L_{P}, which is monitored over time. The exponential decrease in vesicle volume can be calculated from video images, and then fit to determine the permeability coefficient (P_{f}) (see, e.g., Bloom et al., 1991; Needham et al., 1996). The permeability coefficient, P_{f}, determined for EO₄₀-EE₃₇ was 2.5±1.2 μm/second, which, when compared with representative vesicles of stearyl-oleoyl-phosphatidylcholine (SOPC) that have P_{f}=23.5±1.7 μm/second from comparable methods, indicates a significant reduction in the permeability of the polymersomes.

The reduced permeability results mainly from the increased hydrophobic thickness. On a per area basis, EO₄₀-EE₃₇ membranes and phospholipid membranes were found to exhibit similar fluctuations in area as understood from the fact that the membranes have a comparable area expansion modulus. Consequently, the ratio of permeabilities largely reflects the relative probability for water to diffuse across the membrane, and the ratio of diffusion times decrease with relative thickness of the hydrophobic core as exp(−d_{OE7}/d_{lipid}). For polymersomes of EO₄₀-EE₃₇, this yields exp(−8 nm/3 nm)=0.07, which is a value close to the measured ratio of permeabilities for these polymersomes versus phospholipid vesicles.

The cross-linked membrane is also permeable to water. Observed volume changes due to an osmolarity difference between the inside and outside of cross-linked polymersomes are very similar to the volume changes of uncross-linked vesicles under the same conditions, suggesting that the permeability of the cross-linked membrane is quite similar to the measured value for the exemplified EO₄₀-EE₃₇ membranes. In addition, cross-linked vesicles can be completely dehydrated in air, without loss of solutes, and rehydration leads to swelling by water permeation through the membrane.

(2) Permeability of the Polymersome to Encapsulated Materials

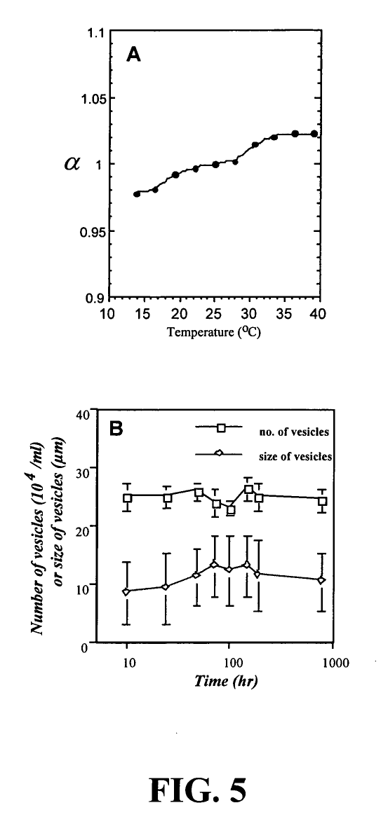

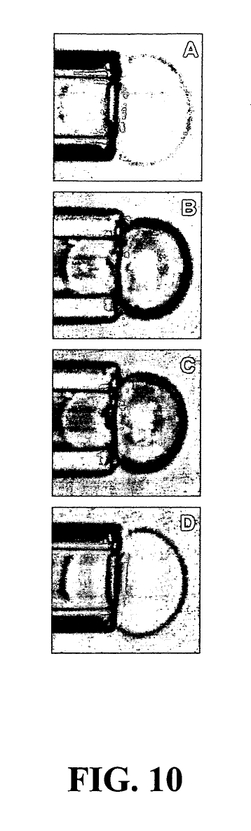

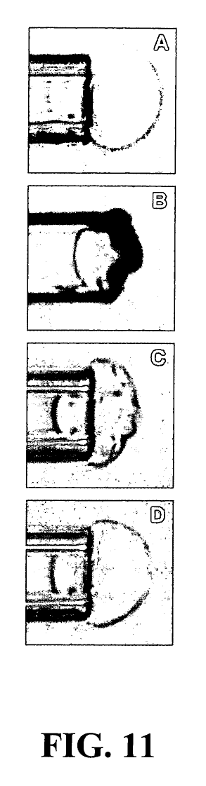

To verify the wide range of molecules encapsulated in the polymersomes, as described above, a method was devised using phase contrast microscopy to give rise to different intensities for materials with distinct optical indices, such as sucrose and phosphate buffered saline. No noticeable change was detected in the intensities or the differences between intensities over time periods from minutes to a month (FIG. 5B). The same was true for the intensities of fluorescently-labeled materials in fluorescent microscopy experiments. Therefore, the polymersome membrane is essentially impermeable to the encapsulated molecules. The impermeability of the cross-linked membrane was also confirmed by the finding that these vesicles retain their encapsulated sucrose, observable through phase contrast, even after complete dehydration and rehydration of the vesicle (FIG. 11), or after 30 minute exposure to chloroform (FIG. 10).

F. Stability of Polymersomes

(1) Stability in Physiological Buffers

FIG. 5B demonstrates the long-term stability of EO₄₀-EE₃₇ polymersomes in phosphate buffered saline. Polymer vesicles were suspended in PBS, and their concentration estimated by counting the intact vesicles using a hemocytometer at different time points. At the same time, the size of the vesicles was determined as an average of twenty randomly selected vesicles. No significant change in the concentration or size distribution of the polymersomes was observed over period of more than one month. Moreover, addition of ethanol to PBS had no significant effect on the polymersome concentration or size distribution, suggesting that such treatments can be use as sterilizing agents (TABLE 3).

(2) Thermal Stability

As shown in TABLE 5, however, the thermal stability of EO₄₀-EE₃₇ vesicles was severely tested when the vesicles were exposed to autoclave temperatures and pressures (121° C., at 2 atm) for 15 minutes. Some vesicles maintained a phase contrast and could be counted as largely retaining their contents. At the dilute polymersome concentrations used in these studies, the results clearly show that a significant fraction (about 10%) of polymersomes can survive a sterilizing treatment such as autoclaving.

TABLE 5
Tabulation of phase dense vesicles after autoclaving

| | Before Autoclave | | After Autoclave | |
|---|---|---|---|---|
| | | Size | | Size |
| | No. of vesicles | distribution | No. of vesicles | distribution |
| Trial # | 10⁴/ml | (μm) | 10⁴/ml | (μm) |
| 1 | 82.4 | 7.3 ± 4.8 | 8.1 | 3.7 ± 0.4 |
| 2 | 94.3 | 6.0 ± 2.8 | 11.9 | 4.0 ± 0.6 |
| 3 | 120.6 | 8.2 ± 5.2 | 10.7 | 3.8 ± 0.5 |

FIG. 5A shows the thermal stability of EO₄₀-EE₃₇ vesicles, indicating the membrane's area expansion with increasing temperature, and its stability at 37° C., when the vesicle is held at a fixed membrane tension of less than 4 mN/m. The relative polymer vesicle area, a, is shown against temperature. The overall thermal expansivity is approximately 1.9×1 0-3 per degree C.

To confirm the thermal stability of the cross-linked polymersomes, the exemplified cross-linked EO₂₆-PD₄₆ vesicles containing an encapsulated 250 mOsm sucrose solution were suspended in 250 mOsm glucose solution. About 0.5 ml of the vesicular solution was added to an Eppendorf test tube and submerged into boiling water for 15 minutes. The number of vesicles before and after boiling was quantified with hemocytometer, and the numbers were found to remain constant at the original level of 10⁵/ml. Thus, the cross-linked EO₂₆—PD₄₆ vesicles are thermally stable at 100° C. for at least 15 minutes. Moreover, the increase in temperature to 100° C. did not alter the phase contrast image of the encapsulated sucrose, confirming that the impermeability of the polymersome membrane is retained at temperatures as high as 100° C.

(3) Stability in Organic Solvents

To confirm the stability of the polymersomes in organic solvents, the exemplified cross-linked EO₂₆-PD₄₆ vesicles were inserted into one of the copolymer's best solvents, chloroform, and observed. Insertion of vesicles into a droplet of chloroform carefully placed in the micromanipulation chamber altered neither the vesicle's size, nor its shape, and the vesicle membrane remained stable for as long as it was kept in the solvent (up to 30 minutes) (FIG. 10). Small, scattering objects appeared inside the cross-linked vesicles when they were placed in contact with chloroform (FIGS. 10B and 10C). However, the particles disappeared when the vesicle was returned to aqueous solution (FIG. 10D). The scattering objects simply indicate, most likely, a finite permeability of the membrane to chloroform and formation of an encapsulated chloroform-in-water microemulsion. Moreover, examination of the vesicles under phase contrast microscopy directly confirmed that they retain large solute molecules, such as sucrose, which also has a significant solubility in chloroform (approximately millimolar).

By contrast, uncross-linked vesicles ruptured, even before they could be transferred by micropipette into the chloroform droplet. This is because the small solubility of chloroform in water (about 0.5% by volume) leads to a concentration gradient near the interface, and even this small chloroform concentration several microns away from the interface, is sufficient to selectively disrupt an uncross-linked vesicle.

(4) Stability to Dehydration and Rehydration

An additional stability test was conducted to confirm the remarkable stability of the cross-linked polymersomes to dehydration. Due to the non-zero permeability of the cross-linked EO₂₆-PD₄₆ vesicles to water, these vesicles can be completely dehydrated in a test tube. The dry vesicles can be stored in air at room temperature for more than 24 hours and then rehydrated by addition of water to restore the vesicle to its original volume. No noticeable difference between the original and rehydrated vesicles was been found.

Individual vesicles can be also aspirated into a micropipette and pulled from aqueous solution into the open air (FIG. 11). As the water evaporates, the volume of the vesicle decreases, and the membrane collapses. The semi-dehydrated vesicle can be inserted back into aqueous solution and rehydrated to its original shape. Phase contrast microscopy confirmed that the encapsulated material, such as sucrose, remains inside the dry vesicles. Therefore, the vesicles can be used in applications that require long-term storage of material.

It is clear from the foregoing, that polymersomes are particularly useful for the transport (either delivery to the bulk or removal from the bulk) of hormones, proteins, peptides or polypeptides, sugars or other nutrients, drugs, medicaments or therapeutics, including genetic therapeutics, steroids, vitamins, minerals, salts or electrolytes, genes, gene fragments or products of genetic engineering, PKH fluorescent dyes, fluorinated lipids, fluorescent-dye modified copolymers, dyes, adjuvants, biosealants and the like. In fact, the stable vesicle morphology of the polymersome may prove particularly suited to the delivery of biosealants to a wound site. In bioremediation, the polymersomes could effectively transport waste products, heavy metals and the like. In electronics, optics or photography, the polymersomes could transport chemicals or dyes. Moreover, these stable polymersomes may find unlimited mechanical applications including insulation, electronics and engineering.

In addition, the polymersome vesicles are ideal for intravital drug delivery because they are biocompatible; that is they contain no organic solvent residue and are made of nontoxic materials that are compatible with biological cells and tissues. Thus, because they can interact with plant or animal tissues without deleterious immunological effects, any drug deliverable to a patient could be incorporated into a biocompatible polymersome for delivery. Adjustments of molecular weight, composition and polymerization of the polymer can be readily adapted to the size and viscosity of the selected drug by one of ordinary skill in the art using standard techniques, so long as the controlled rate of release from the polymersomes of the encapsulant, e.g., the dyes and/or drugs etc is controlled by the blend ratio of the copolymers, copolymer molecular weights, and/or copolymer block ratios (i.e., the weight fraction, f_{EO} of the polyethylene oxide; see Example 5).

Additional encapsulation applications that involve incorporation of hydrophobic molecules in the bilayer core include, e.g., alkyd paints and biocides (e.g., fungicides or pesticides), obviating the need for organic solvents that may be toxic or flammable. Polymersomes also provide a controlled microenvironment for catalysis or for the segregation of non-compatible materials.

The vesicles of the present invention further provide useful tools for the study of the physics of lamellar phases. At different temperatures or reduced volumes (achieved by deflating the vesicle interior with an external high salt solution), such vesicles will display a variety of shapes. The formation of these shapes is dictated by the minimization of energy of deformation of the vesicle, namely the curvature and area elasticity of the membrane. In fact, a series of theoretical models, called “area-difference elasticity” (ADE) models, have been used to predict a limited spectrum of different shapes seen with vesicles, such as buds, pear-shaped vesicles and chains. Comparison between observed shapes and theoretical calculations are used to verify theoretical concepts of how lamellar phases behave, e.g., features such as the curvature, or the tendency of molecules to “flip-flop” between monolayers.

In addition, polymersomes have a small negative buoyancy making them subject to gravitational shape deformations. Therefore, polymersomes afford interesting models for studying the effects of gravitation, or the lack thereof.

The present invention is further described in the following examples. These examples are not to be construed as limiting the scope of the appended claims.

### EXAMPLES

### Example 1

### Polymersomes from Amphiphilic Diblock Copolymers

Membranes assembled from a high molecular weight, synthetic analog (a super-amphiphile) are exemplified by a linear diblock copolymer EO₄₀-EE₃₇. This neutral, synthetic polymer has a mean number-average molecular weight of about 3900 g/mol mean, and a contour length ˜23 μm, which is about 10 times that of a typical phospholipid acyl chain (FIG. 1A). The polydispersity measure, M_{w}/Mₙ, was 1.10, where M_{w} and Mₙ are the weight-average and number-average molecular weights, respectively. The PEO volume fraction was f_{EO}=0.39, per TABLE 1.

Adapting the electroformation methods of Angelova et al., 1992, a thin film (about 10 nm to 300 μm) was prepared. Giant vesicles attached to the film-coated electrode were visible after 15 to 60 min. These were dissociated from the electrodes by lowering the frequency to 3 to 5 Hz for at least 15 min and by removing the solution from the chamber into a syringe. The polymersomes were stable for at least month if kept in vial at room temperature. The vesicles also remained stable when resuspended in physiological saline at temperatures ranging from 10E to 50EC.

Images were obtained with a JEOL 1210 at 120 kV using a nominal underfocus of 6 μm and digital recording. Imaging of the hydrophobic cores of these structures revealed a core thickness d=8 nm, which is significantly greater than d=3 nm for phospholipid bilayers as described in the Handbook of Biological Physics, 1995.

Thermal undulations of the quasi-spherical polymersome membranes provided an immediate indication of membrane softness (FIG. 2A). Furthermore, when the vesicles were made in the presence of either a 10-kD fluorescent dextran (FIG. 2B), sucrose or a protein, such as globin, the probe was found to be readily encapsulated and retained by the vesicle for at least several days. The polymersomes further proved highly deformable, and sufficiently resilient that they could be aspirated into micrometer-diameter pipettes (FIGS. 2C and 2D). The micromanipulations were done with micropipette systems as described above and analogous to those described by Longo et al., 1997 and by Discher et al., Science 266:1032 (1994).

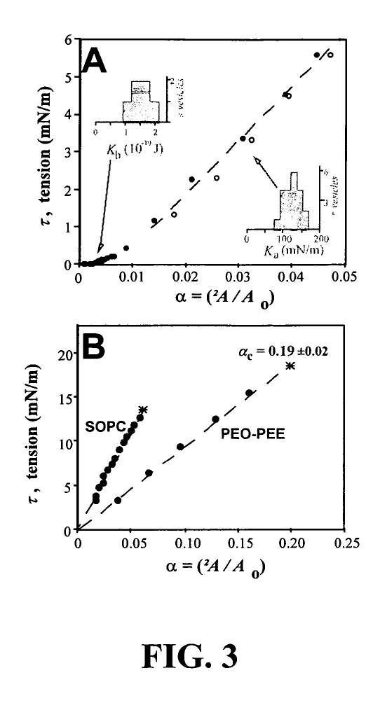

The elastic behavior of a polymersome membrane in micropipette aspiration (at ˜23° C.) appeared comparable in quality to a fluid-phase lipid membrane. Analogous to a lipid bilayer, at low but increasing aspiration pressures, the thermally undulating polymersome membrane was progressively smoothed, increasing the projected area logarithmically with tension, τ, (FIG. 3A). From the slope of this increase (in tension units of mN/m) versus the fractional change, α, in vesicle area the bending modulus, K_{b}, was calculated (see, e.g., Evans et al., Phys. Rev. Lett. 64:2094 (1990); Helfrich et al., Nuovo Cimento D3:137 (1984)).
K_{b}≈k_{B}T ln(τ)/(8πα)+constant Eq (1)

When calculated, it was found to be 1.4±0.3×10⁻¹⁹ Joules (J), based upon the measurements of six vesicles. In equation 1, kB is Boltzmann's constant and T is an absolute temperature. Above a crossover tension, τₓ, an area expansion modulus, Kₐ, was estimated with
Kₐ=τ/α Eq (2)
applied to the slope of the aspiration curve as illustrated in FIG. 3.

Aspiration in this regime primarily corresponds to a true, as opposed to a projected, reduction in molecular surface density, and for the polymersome membranes, Kₐ=120±20 mN/m (based upon 21 vesicles). Fitted moduli were checked for each vesicle by verifying that the crossover tension, τₓ=(Kₐ/K_{b})(k_{B}T/8π), (Evans et al., 1990) suitably fell between appropriate high-tension (membrane stretching) and low-tension (membrane smoothing) regimes.

Measurements of both moduli, Kₐ and K_{b}, were further found to yield essentially unimodal distributions with small enough standard deviations (approximately 20% of mean) to be considered characteristic of unilamellar polymer PEO-PEE vesicles. Interestingly, the moduli are also well within the range reported for various pure and mixed lipid membranes. SOPC (1-stearoyl-2-oleoyl phosphatidylcholine) in parallel manipulations was found, for example, to be approximately Kₐ=180 mN/m (FIG. 3B) and K_{b}=0.8×10⁻¹⁹J. Lastly, at aspiration rates where projection lengthening was limited to <1 μm/s, the microdeformation proved largely reversible, consistent again with an elastic response.

The measured Kₐ is most simply approximated by four times the surface tension, γ, of a pure hydrocarbon-water interface (γ=20 to 50 mJ/m²), and thus reflects the summed cost of two monolayers in a bilayer (see, e.g., Israelachvili, in Intermolecular and Surface Forces, 2^{nd} ed., Sec. III, 1995). The softness of Kₐ compared with gel or crystalline states of lipid systems is further consistent with liquid-like chain disorder as described by Evans et al., 1987. Indeed, because the average interfacial area per chain, <A_{c}>, in the lamellar state has been estimated to be <A_{c}>/2.5 nm² per molecule (see, e.g., Hajduk et al., 1998; Warriner et al., Science 271: 969 (1996); Yu et al., 1998), the root-mean-squared area fluctuations at any particular height within the bilayer can also be estimated to be, on average, <δA_{c}²>^{1/2}=(<A_{c}>k_{B}T/Kₐ)²/0.3 nm² per molecule, which is a significant fraction of <A_{c}> and certainly not small on a monomer scale.

Moreover, presuming in the extreme, a bilayer of unconnected monolayers d/2 thick, with d estimated from cryo-TEM (FIG. 1), the PEE contour length is more than twice the monolayer core thickness, and therefore, configurationally mobile along its length. In addition, molecular theories of chain packing in bilayers have suggested that, although at a fixed area per molecule there is a tendency for K_{b} to increase with chain length (that is, membrane thickness), other factors such as large <A_{c}> can act to reduce K_{b} (see, e.g., Szleifer et al., Phys. Rev. Lett. 60:1966 (1988); Ben-Shaul, in Structure and Dynamics of Membranes from Cells to Vesicles, in Handbook of Biological Physics, vol. 1, chap. 7 (Elsevier Science, Amsterdam, 1995)). Thus, despite the large chain size of EO₄₀-EE₃₇, a value of K_{b} similar to that of lipid bilayers is not surprising.

Related to the length scales above, the root ratio of moduli, (K_{b}/Kₐ)^{1/2}, is generally recognized as providing a proportionate measure of membrane thickness (see, e.g., Handbook of Biological Physics, supra; Bloom et al., 1991; Needham et al., 1996, chap. 9; and Petrov et al., Prog. Surf. Sci. 18:359 (1984)). For the presently described polymersome membranes, (K_{b}-Kₐ)^{1/2}=1.1 nm on average. By comparison, fluid bilayer vesicles of phospholipids or phospholipids plus cholesterol, have reported a ratio of (K_{b}/Kₐ)^{1/2}=0.53 to 0.69 nm (Evans et al., 1990; Helfrich et al., 1984). Typically, the fluid bilayer vesicles of phospholipids plus cholesterol have a higher Kₐ than those of phospholipid alone.

A parsimonious continuum model for relating such a length scale to structure is based on the idea that the unconnected monolayers of the bilayer have, effectively, two stress-neutral surfaces located near each hydrophilic-hydrophobic core interface (see e.g., Petrov et al., Prog. Surf. Sci. 18:359 (1984)). If one assumes that a membrane tension resultant may be located both above and below each interface, then
(K_{b}/Kₐ)=δ_{H}δ_{C} Eq (3)
where δ_{H} and δ_{C} are, respectively, distances from the neutral surfaces into the hydrophilic and hydrophobic cores.

For lipid bilayers with d/2=1.5 nm and hydrophilic head groups equal to 1 nm thick, estimates of δ_{C}=0.75 mm and δ_{H=}0.5 nm yield a root-product, (δ_{H}δ_{C})^{1/2}=0.61 nm. This is consistent with experimental results. The numerical result for PEO-PEE membranes (1.1 nm) suggests that the stress resultants are centered further from the interface, but not necessarily in strict proportion to the increased thickness or the polymer length.

Elastic behavior terminates in membrane rupture at a critical tension, τ_{c}, and areal strain, a α_{c}. With lipids, invariably α_{c}=0.05. This is consistent, it appears, with a molecular theory of membranes under stress (see, e.g., Netz et al., Phys. Rev. E 53:3875 (1996) describing self-consistent calculation models of lipids). For the polymersomes, cohesive failure occurred at α_{C}=0.19±0.02 (FIG. 3B).

Another metric is the toughness or cohesive energy density that, for such a fluid membrane, is taken as the integral of the tension with respect to area strain, up to the point of failure:
E_{c}=1/2Kₐα_{c}² Eq (4)

For a range of natural phospholipids mixed with cholesterol, the toughness has been systematically measured, with E_{c} ranging from 0.05 to 0.5 mJ/m² (see, Needham et al., 1990). By comparison, the EO₄₀-EE₃₇ membranes are 5 to 50 times as tough, with E_{c}≈2.2 mJ/m². On a per molecule, as opposed to a per area basis, such critical energies are remarkably close to the thermal energy, k_{B}T, whereas such an energy density for lipid bilayers is a small fraction of k_{B}T. This difference indicates, that for this relatively simple condensed matter system, the strong role that fluctuations in density have in creating a lytic defect.

Despite the comparative toughness of the polymersome membrane, a core “cavitation pressure,” p_{c}, may be readily estimated as:
p_{c}=τ_{c}/d Eq (5)
yielding a value of p_{c}=−25 atm. This value falls in the middle of the range noted for lipid bilayers, p_{c}=−10 atm to −50 atm (see, e.g., Bloom et al., 1991; Needham et al., 1996). Bulk liquids, such as water and light organics, are commonly reported to have measured tensile strengths of such a magnitude, as may be generically estimated from a ratio of nominal interfacial tensions to molecular dimensions (that is, ˜γ/d). In membrane systems, this analogy again suggests an important role for density fluctuations, which are manifested in a small Kₐ, and which must become transversely correlated upon coalescing into a lytic defect.

Because the previous estimate for <δA_{c}²>1/2 is clearly not small as compared with the cross section of H₂O, a finite permeability of the polymersome membranes to water was expected. To verify this expectation polymersome permeability was obtained by monitoring the exponential decay in EO₄₀-EE₃₇ vesicle swelling as a response to a step change in external medium osmolarity. Vesicles were prepared in 100 mOsm sucrose solution to establish an initial, internal osmolarity, after which they were suspended in an open-edge chamber formed between cover slips and containing 100 mOsm glucose. A single vesicle was aspirated with a suction pressure sufficient to smooth membrane fluctuations; after which the pressure was lowered to a small holding pressure.

With a second, transfer pipette, the vesicle was moved to a second chamber with 120 mOsm glucose. Water flowed out of the vesicle due to the osmotic gradient between the inner and outer surfaces, which led to an increased projection length that was monitored over time. The exponential decrease in vesicle volume was calculated from video images, and then fit to determine the permeability coefficient (P_{f}) (see, e.g., Bloom et al., 1991; Needham et al., 1996). The permeability coefficient, P_{f}, was 2.5±1.2 μm/s.

In marked contrast, membranes composed purely of phospholipids with acyl chains of approximately 18 carbon atoms typically have permeabilities in the fluid state at least an order of magnitude greater (25 to 150 μm/s). Polymersomes are thus significantly less permeable to water, which suggests beneficial applications for the polymersomes.

### Example 2

### Crosslinked Polymersomes

Given the flexibility of copolymer chemistry, the stealth character as well as the cell stability can be mimicked with amphiphilic diblock copolymers that have a hydrophilic fraction comprising PEO, and a hydrophobic fraction which can be covalently cross-linked into a network. One example of a diblock copolymer having such properties, along with the capability of forming several morphologically different phases, is polyethylene oxide-polybutadiene (PEO-PBD).

EO₂₆-BD₄₆, spontaneously forms giant vesicles as well as smaller vesicles in aqueous solutions without the need of any co-solvent. Cross-linkable unilamellar vesicles were fabricated. The formed vesicles were cross-linked by free radicals generated with an initiating K₂S₂O₈ and a redox couple Na₂S₂O₅/FeSO₄.7H₂O as described above. When the osmolarity of the cross-linking reagents was kept the same as that of the vesicle solution, neither addition of the cross-linking reagents nor the cross-linking reaction itself affected vesicle shape.

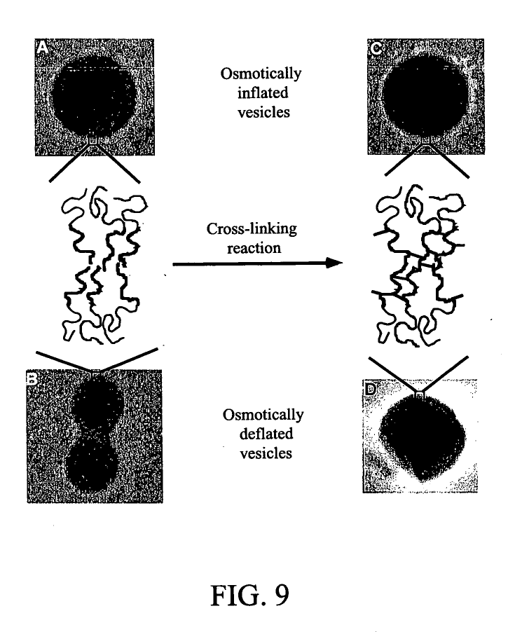

Osmotically inflated vesicles remained spherical, independent of the cross-linked state of the membrane (FIGS. 9A and 9C). Consequently, the fully inflated spheres, pearls of interconnected spheres, and other shapes appeared unchanged from the way they were observed prior to the cross-linking reaction. When fluid phase vesicles are osmotically deflated, the result is a flaccid shape, with a smooth contour (FIG. 9B). However, when the cross-linked vesicles were osmotically deflated after the cross-linking reaction was completed, the vesicles revealed the solid character of the membrane—with irregularly deformed creased structures (FIG. 9D). The difference reflected the fact that, when exposed to a change in osmolyte, the cross-linked molecules could not significantly rearrange within their surface to relax the accumulated strain.

The cross-linked EO₂₆-BD₄₆ vesicles were initially tested for stability by direct observation of the vesicles added into a solvent, i.e., chloroform. However, chloroform altered neither the size, nor the shape of the vesicles, and the vesicle membrane remained stable for as long as it was kept in the solvent. The mechanical properties of the vesicle when exposed to solvent are shown in FIG. 10. FIG. 10A depicts a vesicle in aqueous solution being pulled into a micropipette by negative pressure, ΔP. FIG. 10B depicts the same vesicle imaged immediately after being placed into chloroform. After 30 minutes exposure to chloroform, there was no noticeable change observed in the vesicle (FIG. 10C); and the vesicle remained unchanged after it was returned to the aqueous solution (FIG. 10D).

If a significant portion (few weight percent) of the solutes were lost from the vesicle during chloroform exposure, the aspirated projection of the vesicle would have lengthened. However, no detectable change occurred in either surface area or volume. This demonstrated that the cross-linked membrane maintains its integrity when exposed to organic solvent. By comparison, uncross-linked vesicles cannot be exposed without rupture to aqueous solutions containing a saturating concentration of solvent (approximately 0.8 g/dl chloroform).

A second stability test was based upon complete dehydration. Due to the finite water permeability of the cross-linked vesicles, they can be completely dehydrated in a test tube. Dry vesicles were stored in air, at room temperature, for more than 24 hours, then rehydrated by the addition of water to their original volume. However, no noticeable difference between the original and rehydrated vesicles was found.

Individual cross-linked vesicles were also aspirated into a micropipette, pulled from the aqueous solution (FIG. 11A) and exposed to the open air (FIG. 1I B). As the water evaporated and the vesicle dehydrated, the volume decreased, and the membrane crinkled. Nevertheless, when the semi-dehydrated vesicle was returned to the aqueous solution, it was immediately rehydrated to its original shape (FIG. 11C). Within 1 minute of rehydration, the original shape of the dehydrated vesicle was almost completely restored, indicating the retention of solutes within the vesicle. Phase contrast microscopy further confirmed that encapsulated material, such as sucrose, remained inside the dry vesicles. Therefore, the cross-linked vesicles can be used in applications that require long-term storage of material.

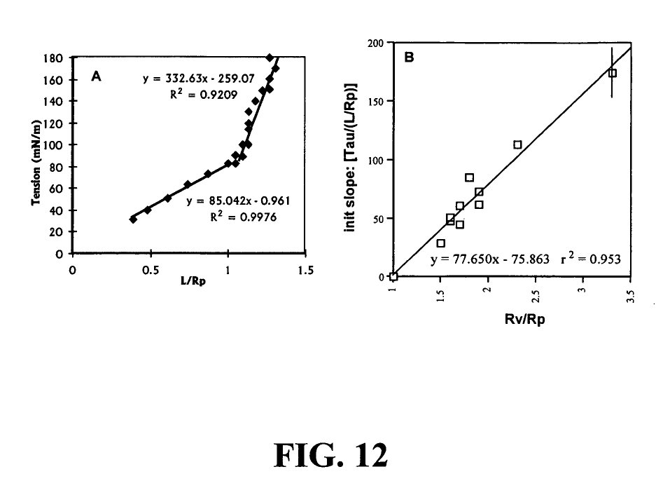

To finally confirm the stability of the cross-linked vesicles, deformation tests were done by micropipette manipulation (FIG. 12). The maximum applied aspiration pressure in the experimental setup, ΔP=1 atm, did not lead to rupture of the cross-linked vesicles. Since the typical micropipette radius in the experiment was 4 μm, such high pressures led to membrane tension at the cap, τ=1/2 ΔPRₚ of around 200 mN/m, which is an order of magnitude higher than the lysis tension of red blood cells. A typical aspiration curve of a flaccid, nearly spherical (but not pressurized) vesicle is shown in FIG. 12A. Such aspiration curves can be done repeatedly, indicative of the membrane's elasticity.

Since the aspirated vesicles were flaccid, but almost spherical and non-pressurized, it was assumed that during initial aspiration, the area of the vesicle is constant, and that the bending becomes negligible with respect to shearing of the membrane. Given those assumptions, computer simulations for the shearing of the vesicle in the pipette indicated that the shear modulus is between one and two times the slope of τ/(L/Rₚ) versus Rᵥ/Rₚ (FIG. 12B). This was equal to about 150 mN/m, which is four orders of magnitude higher than the shear modulus of red blood cells, which was determined to be about 0.01 mN/m.

Although proving that a membrane is completely cross-linked is not a trivial task, and controversy is often associated with the subject, the stability tests reported in the present example provide the best direct evidence to date to confirm complete cross-linking. Cross-linking reactions introduce local stresses in the membrane, making it more difficult to completely cross-link a large (cell-size) structure that is self-assembled from monomers with a limited number of cross-linkable entities. However, by expanding the size of the polymerizable block in the present invention, the difficulties have been overcome.

### Example 3

### Polymersomes from Amphiphilic Triblock and Multi-Block Copolymers

Multi-block copolymers offer an alternative approach to modifying the properties of the polymersome. Insertion of a middle B block in a triblock copolymer permits modification of permeability and mechanical characteristics of the polymersome without chemical cross-linking. For example, if the B and C blocks are strongly hydrophobic, yet mutually incompatible, and the A block is water miscible, two segregated layers will form within the core of the membrane. This configuration of interfaces (internal B-C and external B-hydrated A) offers control of the spontaneous curvature of the membrane among other features such as height-localized cross-linking. Thus, vesicle size will depend, in part, on block copolymer composition. Of course, as noted above, the physical properties of the ABC polymersome will reflect a combination of the B, C and hydrated A mechanical behaviors. An example of such a triblock copolymer, which does form vesicles is EO₃₃-S₁₀-I₂₂ (TABLE 1), wherein EO is polyethyleneoxide, S is styrene, and I is isoprene.

Another arrangement for the triblock, which would form vesicles, is ABA or ABC wherein A and C are water miscible blocks and B is the hydrophobic block. In such case the copolymer can self-assemble in “straight” form into a monolayer or in “180° bent” form into a bilayer, or as a combination of these two forms. An example of this kind of ABA triblock, which does form vesicles, is EO₄₈-EE₇₅-EO₄₈ (TABLE 1).

### Example 4

### Vesicles of Mixed Composition

Vesicles comprising diblock copolymer mixtures have been prepared by the methods described above for a wide ratio of diverse amphiphilic components. As a first example, mixture of cross-linkable diblock copolymers with noncross-linkable ones can be made. However, in contrast to the stabilizing effect of cross-linking on vesicles fabricated from purely cross-linkable amphiphiles as described above, the dilution of cross-linkable amphiphiles with non-cross-linkable molecules could produce a less stable membrane upon cross-linking, resulting in a controlled-release membrane.

For the purpose of this invention, the percolation threshold is a weight fraction of the cross-linkable copolymer above which the cross-linking reaction leads to a single cross-linked domain spanning the entire vesicle surface. Below the percolation threshold, a single cross-linked domain does not span the entire vesicle surface and is likely to be much less stable than a wholly cross-linked vesicle. For example, mixtures of EO₄₀-EE₃₇ and EO₂₆-PD₄₆ copolymers with the weight fraction of EO₂₆-PD₄₆ equal to 0.5 were found to be extremely fragile after the cross-linking reaction as compared with single component polymersome membranes (and therefore below the percolation threshold).

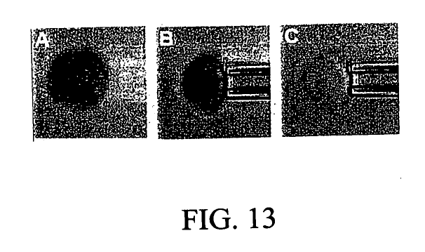

Increase of the weight fraction to 0.6 caused the vesicles to be more stable than the uncross-linked membranes, but far more fragile than the vesicles composed of purely cross-linkable amphiphiles, as demonstrated by the leakage of encapsulated material (FIG. 13). Therefore, appropriate mixing of different components can be used to modulate vesicular stability. The destabilization by this type of cross-linking reaction can be applied to controlling the release of contents from the polymersome vesicle. Consequently, the polymersome can be induced to release an encapsulated component, either chemically and/or by wave propagation (such as, X-rays, UV, visible light, IR irradiation, and ultrasound).

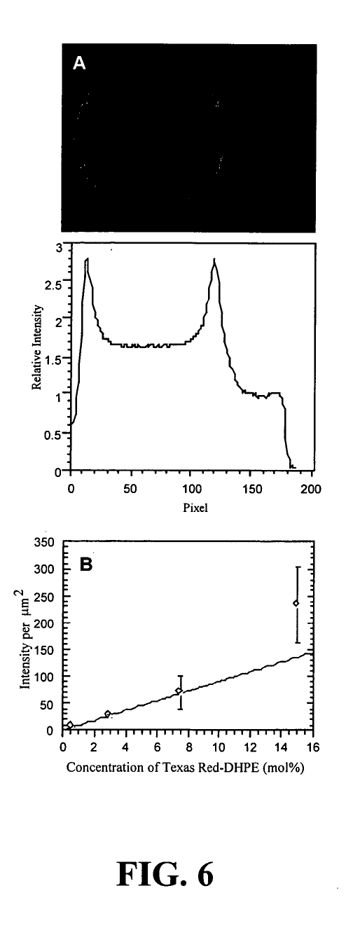

In the same way, mixtures can be made of the copolymer amphiphiles with other synthetic or non-synthetic amphiphiles, such as, lipids or proteins. For example, 3% of a Texas-Red labeled phosphatidylethanolamine preparation was incorporated into an EO₄₀-EE₃₇ membrane with no obvious effect on either membrane structure or area expansion modulus (FIG. 6). FIGS. 6A and 6B show the uniformity of fluorescence around an aspirated contour of membrane with 3 mol % mixed in with polymer before vesicle formation. The uniformity of the fluorescence can be seen around an aspirated contour of the membrane demonstrating good mixing in the membrane.

Moreover, in FIG. 6C the contour intensity was seen to increase linearly as the concentration of Texas Red was increased to about 10 mol %, demonstrating ideal mixing of the components at that concentration range. Laser-photobleaching demonstrates that lipid probe diffusivity is 20-fold lower on average in the polymer membrane than in a lipid (SOPC) membrane which, by the present method has a diffusivity of approximately 3×10⁻⁸ cm²/s.

Based on the above features of amphiphile incorporation into polymersome membranes, the fluorescent lipophilic probe diI(C18) has been incorporated at a few mole percent into cross-linkable membranes and shown to yield unstable membranes after approximately 60 minutes of fluorescence excitation and photo-bleaching.

In sum, polymersomes, enable direct measurements of the material properties of lamellae and permit characterization of membrane assembly. The preparation methods of the present invention provide additional ways to “engineer” bilayer membranes. As compared with lipids, the increased length and conformational freedom of polymer chains of this invention, not only provide a basis for enhanced stability, toughness and reduced permeability of membranes, but also provide a rich diversity of block copolymer chemistries (molecular weights, block fraction, block architecture), thereby furnishing a plethora of novel, artificial membranes and tissues, soft biomaterials and bio-mimetic structures, controlled-release vehicles and systems for engineering and biomedical applications.

### Example 5

### Hydrolysis-Triggered Controlled Release Vesicles

Chemically reactive polyethylene glycol PEG-lipids can play dual roles as liposome stabilizers that also, upon exposure to an environmental stimulus, effectively destabilize the carrier membrane via thiolytic (Kirpotin et al., 1996; Zalipsky et al., 1999) or hydrolytic (Shin et al., 2003; Boomer et al., 2003; Bergstrand et al., 2003) cleavage of their PEG-lipid bonds. As stabilizers, a small percentage (5-10%) of PEG-lipid was found, some time ago, to also delay liposome clearance (Klibanov et al., FEBS Lett. 268:235-237 (1990)). In other words, PEG imparts stealthiness, but until the present invention, neither of the concepts—controlled release or stealth—had been applied to purely synthetic polymer vesicle systems, which permit broad control over vesicle properties.

In this example the polymersomes are composed of block copolymers comprising a combination of both PEG and a hydrolytically susceptible polyester of either polylactic acid (PLA) or polycaprolactone (PCL). Both PLA (Belbella et al., Internat'l J. Pharmaceutics 129:95-102 (1996); Anderson et al., Advanced Drug Delivery Reviews 28:5-24 (1997); Brunner et al., Pharmaceutical Research 16:847-853 (1999); Woo et al., J. Controlled Release 75:307-315 (2001)), and PCL (Pitt in: Langer & Chasin (Eds.), Biodegradable Polymers as Drug Delivery Systems, Marcel Dekker, New York, N.Y., 1990, pp. 71-120; Chawla et al., Internat'l J. Pharmaceutics 249:127-138 (2002)) have been widely studied as readily hydrolysable polyesters. PEG-PLA or PEG-PCL block copolymers are both well known in the art, and their formation is not the subject of this invention (Gref et al., Science 263:1600-1603 (1994); Matsumoto et al., Internat'l J. Pharmaceutics 185:93-101 (1999); Allen et al., J. Controlled Release 63:275-286 (2000); Panagi et al., Internat'l J. Pharmaceutics 221:143-152 (2001); Riley et al., Langmuir 17:3168-3174 (2001); Avgoustakis et al., J. Controlled Release 79:123-135 (2002), herein incorporated by reference). However, recent illustrations of PEG-PLA vesicles (Discher et al., Science 297:967-973 (2002); Meng et al., Macromolecules 36:3004-3006 (2003); Ahmed et al., Langmuir 19:6505-6511 (2003)) highlight the need for detailed characterization and control of release and degradability.

Vesicle formulations of PEG-PLA or PEG-PCL with or without inert PEG-PBD (polybutadiene), a well-documented vesicle former in water (Discher et al., Science, supra, 1999), are shown here to provide programmed control over release kinetics. The dense 100% PEG corona of the PEG-PBD vesicles has recently been shown to deter membrane opsonization, and extend in vivo circulation times significantly beyond stealth liposomes (Photos et al., J. Controlled Release 90: 323-334 (2003)). While broader compatibility of PBD has been explored by others (Kidane et al., Colloids and Surfaces, B, Biointerfaces 18:347-353 (2000); Tseng et al., Biomaterials 16:963-972 (1995)), the in vitro focus here is on the general principle of blending degradable and inert copolymers.

The elusiveness of making PEG-PLA vesicles is largely attributable to limited copolymer designs in relation to narrow requirements for a suitable lamellar phase. Extensive theoretical (Bates, Science, supra, 1991; Fredrickson et al., Physics Today 52:32-38 (1999)), as well as general experimental studies of block copolymer amphiphiles, have established that aggregate morphology, in dilution, is principally determined by molecular geometry.

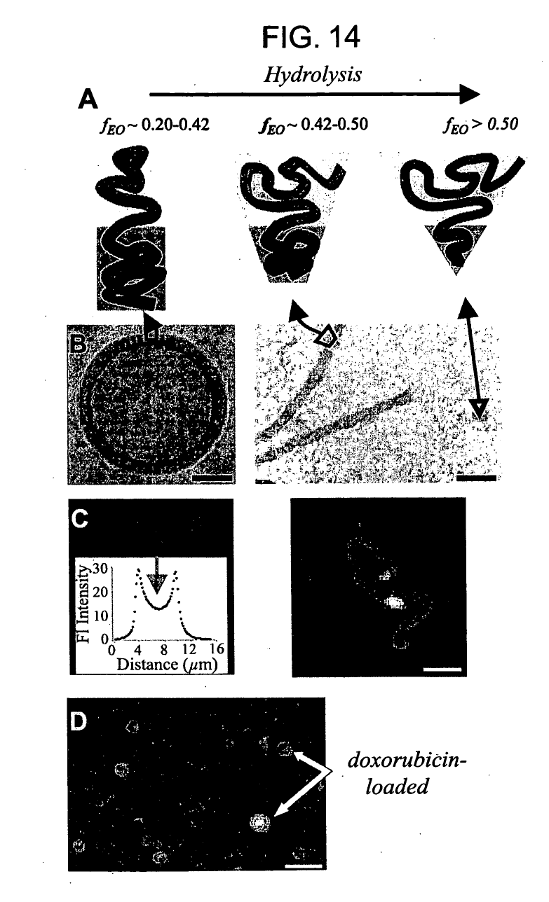

Kinetic traps are many (e.g., entanglements, crystallization, or glassiness at high molecular weight, MW), but when solvated selectively, a delicate, but now relatively well-understood, balance of hydrophilic/hydrophobic segments emerges (FIG. 14A) (Discher et al., 2002; Jain et al., Science 300:460-464 (2003)). This balance allows design of PEG-block based copolymers that, in the absence of degradation, form membranes in preference to other structures. Whereas diblock copolymers with small hydrophilic PEG fractions of f_{EO}<20% and large MW hydrophobic blocks exhibit a strong propensity for sequestering their immobile hydrophobic blocks into solid-like particles (for PEG-PLA (Gref et al., 1994; Avgoustakis et al., 2002; Govender et al., Internat'l J. Pharmaceutics 199:95-110 (2000)), an increased f_{EO}˜20-42% generally shifts the assembly towards more fluid-like vesicles (Discher et al., 2002; Meng et al., 2003; Discher et al., 1999; Nardin et al., Langmuir 16:1035-1041 (2000); Bermudez et al., Macromolecules 35:8203-8208 (2002); Dimova et al., European Physical J., E, Soft Matter 7:241-250 (2002); Checot et al., European Physical J., E, Soft Matter 10:25-35 (2003); Najafi et al., Biomaterials 24:1175-1182 (2003); Valentini et al., Langmuir 19:4852-4855 (2003)), or other “loose” micellar architectures (Piskin et al., J. Biomaterials Science, Polymer Ed. 7:359-373 (1995); Yasugi et al., Macromolecules 32:8024-8032 (1999); Kim et al., Macromolecular Rapid Communications 23:26-31 (2002)). As used herein, “f_{EO}” refers to the hydrophobic to hydrophilic ratio.

For f_{EO}>42%, however, one generally finds both worm micelles (up to ˜50% f_{EO}) (Jain et al., 2003; Won et al., 1999; Dalhaimer et al., Comptes Rendus. Physique 4:251-258 (2003)) and, as noted by others, spherical micelles (for PEG-PLA [Yasugi et al., 1999; Kim et al., 2002); Hagan et al., Langmuir 12:2153-2161 (1996)), and PEG-PCL (Savic et al., Science 25:615-618 (2003)). Lastly, although kinetic traps to equilibrium may deepen with molecular weight (MW), the equilibrium boundaries enumerated above between predominant microphases are only weakly dependent on MW. Recent work indeed shows that the aforementioned f_{EO} values decrease for diblocks only by about 5-6% per addition of 100 EO monomers (Jain et al., 2003).

Nonetheless, while vesicle/micelle transition mechanisms have been exploited in otherwise conventional liposomal systems (Adlakha-Hutcheon et al., 1999; Holland et al., Biochemistry 35(8):2610-2617 (1996); Zhigaltsev et al., Biochim. Biophys. Acta 1565:129-135 (2002); Guo et al., Biophysical J. 84:1784-1795 (2003)), the kinetic aspects of phase transitions have not been easily predicted. Yet, they are of paramount importance when using ‘active’ chains, such as the hydrolytically degradable PEG-PLA for release mechanisms. Considerable data in the literature indicate that degradation of PLA nanoparticles occurs on the order of weeks (Belbella et al., 1996; Piskin et al., 1995). By comparison, in the vesicles of the present invention, it is shown that tunable, controlled release, ranging from hours to many days, results from copolymer blending within the membrane, as well as polyester selection and chain architecture (i.e., f_{EO}).

Materials and Methods

1. Copolymers and Chemicals

The diblocks listed in TABLE 6, except for OB18 and OL1, were purchased from Polymer Source (Dorval, Quebec, Canada). Note that EO denotes ethylene oxide, and that polyethylene oxide is structurally the same as PEG (polyethylene glycol). Tetramethylrhodamine-5-carbonylazide (TMRCA) was obtained from Molecular Probes (Eugene, Oreg.); dialysis tubing and dram vials were from Spectrum Laboratories (Rancho Dominguez, Calif.) and Fisher Scientific (Suwanee, Ga.), respectively. L-Lactide, mono-methoxy polyethylene glycol, tin ethyl hexanoate, toluene, chloroform, methylene chloride, sucrose, dextrose, phosphate buffer (PBS), doxorubicin, and fluorescent dextrans were all purchased from Sigma (St. Louis, Mo.).

2. Synthesis of the Diblock Copolymers

The PEG-PBD diblock (OB18) was synthesized by an anionic polymerization technique described elsewhere (Hillmyer et al., Macromolecules, supra, 1996, herein incorporated by reference). Diblock copolymers, listed in TABLE 6, were synthesized by standard ring opening polymerization detailed below for the PEG-PLA diblock, OL1. Briefly, OL1 used L-lactide and methoxy polyethylene glycol, which were pre-purified by recrystallization from ethyl acetate and toluene, respectively.

The catalyst, tin ethyl hexanoate was used without further purification. All reagents were dissolved in toluene solvent and placed in a sealed pressure tube under argon atmosphere, due to the sensitivity of the lactide monomer to degradation. The reaction vessel was placed in an oil bath at 100° C., and polymerization was allowed to proceed for 2 hours. Polymerization was terminated with a 10-fold excess of hydrochloric acid, and the polymer was further washed in ice-cold cyclohexane. The final product was subsequently lyophilized into a white powder and, when needed, solubilized in chloroform.

¹H NMR was used to determine the number of monomer units in each block. Gel permeation chromatography was used to determine the total number-average molecular weights, Mn, as well as the polydispersity indices (PD). Moreover, preliminary separations after base-catalyzed hydrolysis (pH >12) demonstrated that these synthetic diblocks undergo complete degradation in ≦24 h. The PEG volume fraction (f_{EO}) was converted from the measured mass fractions by using homopolymer melt densities: 1.13, 1.09, 1.14, and 1.06 g/cm³ of PEG, PLA, PCL, and PBD, respectively.

3. Characterization of OL1 Vesicles

Vesicles of pure OL1 block copolymer were prepared by dissolving polymer at 1 wt % in water. The solution was stirred for at least 6 hours at room temperature, and OL1 vesicles were observed by cryogenic transmission electron microscopy (cryo-TEM) (Lin et al., J. Phys. Chem. 97:3571(1993)).

TABLE 6
Physical properties of the various diblock copolymers

| Copolymer | Formula | | | | |
|---|---|---|---|---|---|
| name | Am − Bn | Mₕ^{a} (kg/mol) | Mₙ (kg/mol) | P.D. | f_{EO} |
| OL1 | EO₄₃ − LA₄₄ | 3.2 | 6.0 | 1.1 | 0.33 |
| OL2 | EO₁₀₉ − LA₅₆ | 4.0 | 10.0 | 1.16 | 0.49 |
| OCL1 | EO₄₆ − CL₂₄ | 2.7 | 4.77 | 1.19 | 0.42 |
| OCL2 | EO₁₁₄ − CL₁₁₄ | 12.9 | 18.0 | 1.50 | 0.28 |
| OB18 | EO₈₀ − BD₁₂₅ | 6.8 | 10.4 | 1.1 | 0.29 |

^{a}Mₕ ˜ n × Mₘₒₙₒₘₑᵣ

Briefly, samples of the polymer solution were immersed in a microperforated grid under controlled temperature and humidity conditions. The assembly was then rapidly vitrified with liquid ethane, and kept under liquid nitrogen until loaded onto a cryogenic sample holder. Images (FIG. 14B) were obtained with a JEOL 1210 TEM at 120 kV using a magnification of 20,000 along with a nominal under focus for im-proved resolution and digital recording.

4. Labeling of PEG-PLA (OL1) Block Copolymer

Since the PEG block of the OL1 and OL2 block copolymer was protected with a methoxy group, only the hydroxyl end group of the PLA block was susceptible to modification with tetramethyl rhodamine-5 carbonyl azide (TMRCA; MW 455.5 Da). The modification involved TMRCA conversion to an isocyanate, which then modified the hydroxyl end group to a urethane. This end-group modification, using a 1:1 polymer to dye mole ratio, was carried out overnight in a mixture of toluene and methylene chloride (2:1 v/v) at 60° C. The reaction was carried out in an organic phase primarily to minimize hydrolysis of the PLA block. Excess, unreacted TMRCA dye was dialyzed (MWCO 3500) into chloroform for 1 week, and the labeled block copolymer was stored at 4° C.

5. Preparation of Polymer Bilayers and Encapsulant Loading

Polymer blends with OB18 and either OL or OCL block copolymer were prepared by first solubilizing the polymers at desired molar ratios in chloroform. The organic solvent was then evaporated under nitrogen, followed by vacuum drying for 7 hours to remove trace amounts of chloroform as the polymer film dried onto the glass wall of a dram vial. The film was subsequently hydrated with solutions of hydrophilic encapsulants (active agents), such as sucrose, fluorescently tagged dextrans, or ammonium sulfate (for subsequent doxorubicin loading, below). Thus, the polymersomes of the present invention a simultaneously formed and loaded with encapsulant, or the polymersomes are first formed (“empty”) and subsequently “loaded” with encapsulant. Either can result, however, in a loaded polymersome. Upon hydration, vesicle self-assembly was further promoted in a 60° C. oven for ˜12 hours. Doxorubicin loading was achieved after vesicle formation by a variation of the ammonium sulfate-driven permeation method of Haren and Barenholz et al. (Biochim. Biophys. Acta 1151:201-215 (1993), herein incorporated by reference).

Unencapsulated ammonium sulfate was removed by dialysis (cutoff 3.5 kDa) into isotonic PBS. The drug was added to the vesicle suspension with membrane permeation and accumulation promoted by the species gradients between inside and out of the vesicles. A 10-hour incubation at 37° C., followed by 10-hour dialysis into PBS, proved sufficient for doxorubicin loading, based on both fluorescence microscopy and spectrofluorimetry.

6. Vesicle Isolation and NMR Analysis

Polymer films of pure OB18, OL2, and OL2/OB18 at 50:50 blend ratio were prepared as above, using deuterated water (D₂O). Vesicle blends were separated from free monomers and other small aggregates by extensive dialysis (cut-off ˜1 MDa). Post-dialysis, the polymer solution was thoroughly dried using a rotavap. Pure and 50:50 blend films were subsequently dissolved in CDCl₃ for room temperature ¹H NMR analysis (Astra500 spectrometer, 500 MHz).

7. In Vitro Release Kinetics

Micron-sized vesicles loaded with hydrophilic encapsulants were suspended in PBS (pH 7.0; 300 mosM) and incubated in a closed chamber formed with a gasket seal between a bottom cover slip and a top glass slide (height ˜100 μm). Vesicles were imaged with either bright field or phase contrast using a Nikon TE-300 inverted microscope. Phase contrast microscopy was possible because of the differences in the refractive indices of the encapsulant and the external buffer solution (e.g., sucrose inside and PBS outside). In vitro release kinetics were monitored over time by quantifying the population of vesicles that either retained (“loaded”) or released (“empty”) lumenal encapsulants. An average of 150-300 giant vesicles of various sizes were monitored over the time course of the experiment.

Results

1. PEG-PLA Vesicles and Blends

Both PLA and PCL are generally considered hydrophobic provided they are of sufficiently high molecular weight (Discher et al., 2002). The spontaneous aggregation and assembly of OL1 copolymer (TABLE 6: EO₄₃-LA44) into lamellar or bilayer morphology, i.e., a vesicle, in dilute solution is verified by direct cryo-TEM imaging (FIG. 14B). The hydrophobic core of the membrane provided the contrast and had a measured width equivalent to d≈10.4±1.4 nm.

The miscibility of OL1 block copolymer in a vesicle membrane with OB18 (TABLE 6: EO₈₀-BD₁₂₅) is demonstrated in FIG. 14C by fluorescence microscopy on ‘giant’ vesicles. The hydroxyl end group of the hydrophobic PLA block was first reacted with fluorophore (TMRCA), and the labeled copolymer was then blended in a good solvent with both unlabeled OL1 and OB18 block copolymer at molar ratios of 5:20:75, respectively. Subsequent preparation of a dried film of this blend followed by overnight hydration lead to spontaneous, self-directed assembly of polymersomes that were many microns in diameter. Giant vesicles show similar levels of fluorophore partitioned into the edge-bright membranes (see FIG. 14 inset intensity analysis). A more quantitative analysis of miscibility is provided in the following section. In addition, osmotically driven shape and volume changes of such giant vesicles (Discher et al., 1999) allow visual proof that water necessarily permeates the membrane, which is a pre-requisite for hydrolytic cleavage.

FIG. 14D shows OL1 vesicles stably containing doxorubicin (a widely used anti-tumor therapeutic (Arcamone, Doxorubicin: Anticancer Antibiotics, Academic Press, New York, pp. 126-157 (1981); Kong et al., Cancer Research 60:6950-6957 (2000); Ulbrich et al., J. Controlled Release 87:33-47 (2003)). The result illustrates both the initial integrity and the loading capabilities of the vesicle membranes. The increased membrane thickness of the polymersomes is probably responsible for two to three times longer loading times. Nonetheless, doxorubicin loading proves similar to liposomes (Haren and Barenholz et al., 1993) with roughly 1:1 copolymer: drug (mol/mol) ratios as estimated by spectrofluorimetry. The following sections focus on the encapsulant release of model hydrophilic drugs ranging in molecular weights from ˜10² Da (like doxorubicin) to 10⁵ Da.

2. Miscibility of PEO-PLA in PEO-PBD

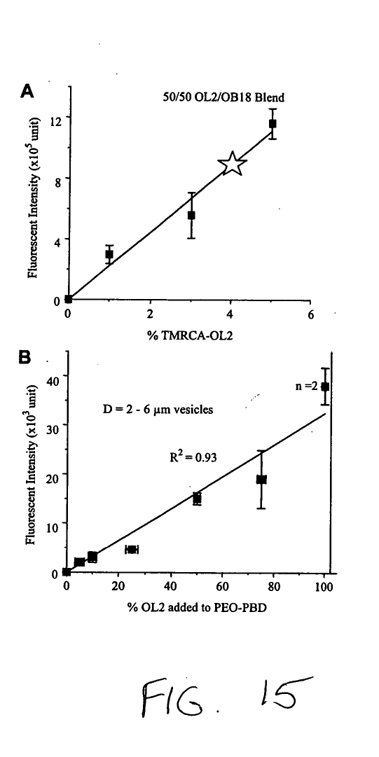

To address block copolymer miscibility in lamellar architectures, such as bilayer vesicles, blends of OL2/OB18 were prepared with fluorescently tagged, TMRCA-OL2 (FIG. 15). To remain within the quenching limit of the fluorophore, varying amounts of TMRCA-OL2 were added to a constant OL2/OB18 blend ratio of 50:50 mol % (FIG. 15A). The fluorescent intensity of the vesicle membrane increased linearly with the added TMRCA-OL2 polymer. Since 4% labeled OL2 provided an adequate signal, it was thus introduced to unlabeled OL2 for blending with OB18 from 5 to 100 mol %.

Upon hydration and self-assembly, vesicle populations were imaged under set conditions of dilution and image collection. Peak or edge intensities of the vesicle membranes were averaged over vesicle diameters ranging from 2 to 6 μm. These intensities appeared to be consistent and reproducible for at least three independent samples prepared over several weeks, indicating stability of the fluorophore conjugate. The clearly linear trend showed that increasing amounts of blended OL2 produced a proportional increase in the intensities of the polymersome membrane.

As a check on the fluorescence imaging results, NMR was done on blended vesicles made with 50:50 OL2/OB18. Analysis of the pure OL2 and OB18 spectra showed the respective peaks for PLA, PEG and PBD, PEG (Riley et al., 2001; Hrkach et al., Biomaterials 18:27-30 (1997); Lucke et al., Biomaterials 21:2361-2370 (2000); Salem et al., Biomacromolecules 2:575-580 (2001); Kukula et al., J. Amer. Chem. Soc. 124:1658-1663 (2002). The nominal 50:50 OL2/OB18 blend appeared to be a summation of the two individual spectra. The mol % OB18 in the blend was derived from the decrease in the integrated intensity ratio normalized to PEG, using the high-ppm OB18 peak in the pure sample [(δ_{PBD,-CH=}=5.29 ppm: I_{5.29} ppm=0.51), (δ_{PEG,CH2}=3.64 ppm: I_{3.64 ppm}=1.0)] versus the blend sample [(δ_{PBD,-CH=}=5.15 ppm:I_{5.15} ppm=0.24), (δ_{PEG,CH2}=3.68 ppm: I_{3.68} ppm=1.0)]. The high ppm peak, thus, had a relative integrated intensity of 0.51 that decreased to 0.24 for the nominal “50:50 blend.” The decrease was due to the PEG contribution from OL2. Accounting for the different PEG chain length allows a straightforward determination of the actual blend ratio as (OL2/OB18)=44:56 mol % (from NMR).

Similar analyses of other resonant peaks (e.g., δ_{PBD,=CH2}=4.91 ppm) suggests an error of about 7%. To summarize, the linear increase of fluorescence intensities with blend ratios (FIG. 15) along with the appearance and quantification of characteristic NMR peaks for both copolymers in OL2/OB18 blends provides clear evidence of OL miscibility in OB18 blends.

3. Visualizing Hydrophilic Encapsulant Release

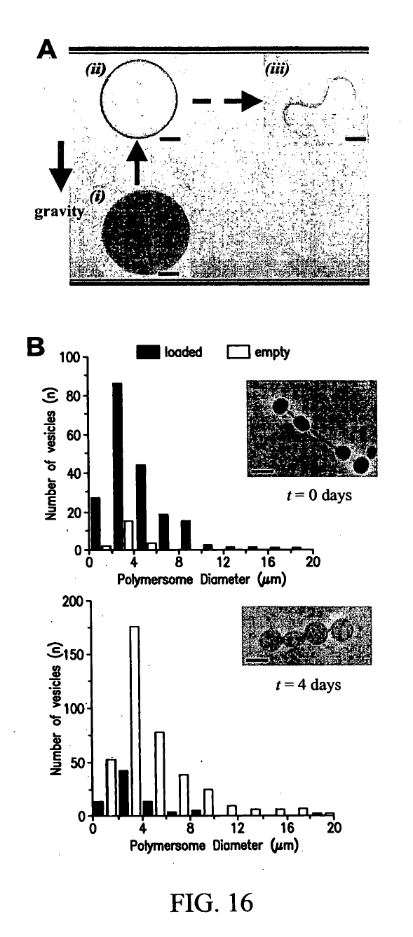

Blends of OL1 or the other degradable diblocks (TABLE 6) with the inert copolymer OB18 have proven to be particularly useful in protracting the time scales for observation of membrane transformation and release processes. For a given blend ratio, vesicles were made in sucrose (see Materials and Methods, supra), a prototypical low molecular weight encapsulant. When diluted into PBS and added to a 100-μm-high sealed chamber for long-term microscopy, vesicles initially settled and appeared dark under phase contrast microscopy (FIG. 16A(i)). This is due to differences in both the specific gravity and the refractive index of the sucrose encapsulant, as compared to the external PBS. Over a span of hours to days in the sealed chamber, a given vesicle will become phase light, buoyant, and rise to the top of the chamber (FIG. 16A(ii)).

Few, if any, vesicles were seen as either half-dark or halfway above the bottom, indicating a two-state system with respect to encapsulant retention, i.e., “loaded” or “empty.” After longer times, the empty vesicles at the top of the chamber, lost their morphology and began to clearly disintegrate in solution (FIG. 16A(iii)). In contrast, pure OB18 vesicles showed essentially no loss of encapsulant over the duration of the study, fully consistent with previous measures of polymersome stability (Lee et al., Biotechnol. Bioengineer. 73:135-145 (2001)).

Histograms of phase contrast vesicles for a given sealed chamber are binned by vesicle size (FIG. 16B), and show clear population shifts from loaded to empty vesicles over hours to days of periodic observation. Since vesicle numbers in all size bins (from 2 to 20 μm) change dramatically over time, the histograms indicate no strong dependence on vesicle diameter. This suggests a surface ‘erosion’ mechanism that occurs locally in the membrane as opposed to a faster process with total degradable mass (which scales as ˜Rᵥₑₛ²). The release studies outlined below demonstrate erosion as a clear poration process with an initial, characteristic pore size.

4. Growth of Membrane Pores of Finite Size

By visually monitoring release from micron-sized vesicles (FIG. 16A(ii)), it is clear that these vesicles retained their overall morphology after releasing their encapsulant. Hydrolysis of the PLA chains in the hydrophobic core of the bilayer is likely to generate some curvature-preferring chains (with f_{EO}=0.42), which localize and induce the growth of pores in the membrane. In order to verify pore induction in the vesicle membrane and provide a gauge for pore size, kinetically tractable 25:75 blends (OL1/OB18) were used for monitoring release profiles of fluorescent dextran encapsulants of 4.4, 66, or 160 kDa dissolved in sucrose. In any given vesicle, it is possible to monitor two labeled dextrans, in addition to sucrose, at the same time by using different fluorophores (e.g., fluorescein or rhodamine).

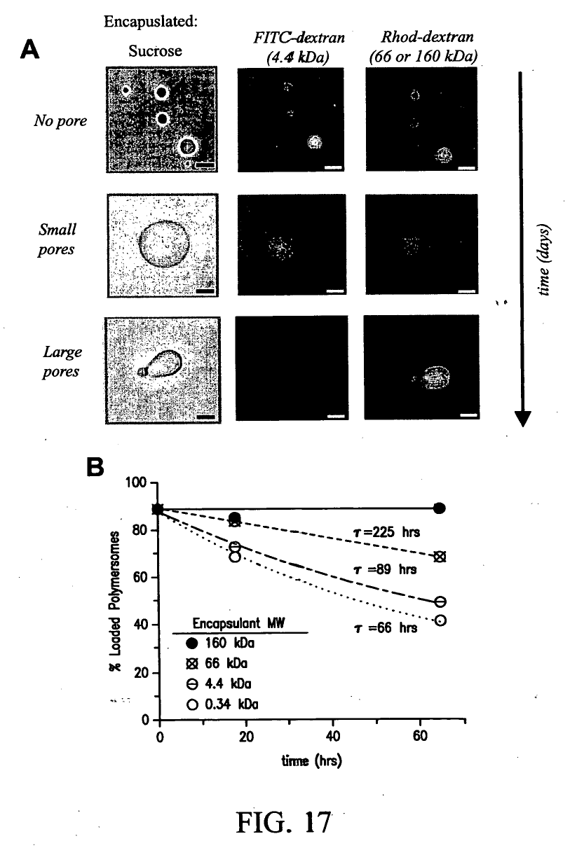

FIG. 17 illustrates the molecular weight dependence of encapsulant release. At initial times (t=0 hour), essentially the entire vesicle population (90-100%) retains all of its encapsulants (i.e., sucrose, 4.4 and 66 kDa dextran). However, by t=18 hours, 22% of the vesicle population has released its encapsulated sucrose. Within this set, nearly two-thirds (15% total) of the vesicles released the 4.4 kDa dextran, and the remaining third (7%) had lost all three of the encapsulants. This data (FIG. 17B) indicates that sucrose and the 4.4 kDa FITC-dextrans were released with respective τᵣₑₗₑₐₛₑ=66 and 89 hours. In contrast, larger molecular weight dextrans (60 kDa) show little to no release from these same carriers until eventual vesicle disintegration occurs on the order of many days.

To attribute a mean length-scale to the transient pore that develops in a vesicle membrane, encapsulant molecular weights were converted (Bu et al., Macromolecules 27:1187-1194 (1994); Hobbie et al., Intermediate Physics for Medicine and Biology, 3rd ed., AIP Press, New York, 1997, pp. 114-124) to mean radii of gyration (R_{g}) with sucrose (0.34 kDa) and dextrans of 4.4, 60, and 160 kDa having respective R_{g}'s of 0.9, 1.4, 4.8, and 7.3 nm. Given the vesicle leakage of all but the last dextran, a conservative upper bound of the hydrophilic pore size was estimated to be 5 nm. This mean radius corresponds to an initial pore diameter of ˜10 nm, which is comparable to the cited membrane thickness of d_{OL1}˜10.4 nm (FIG. 14B), as well as d_{OB18}˜15 nm (Bermudez et al., 2002). Whether or not there is an energetic basis for initial pore size is, at present, unclear.

As a more physical demonstration of carrier instability, the mechanical integrity of blended polymersome vesicles was tested by micropipette aspiration (not shown). Aspiration of an encapsulant loaded vesicle yields rupture strains of the same order of magnitude as pure OB18 vesicles (Bermudez et al., 2002). In marked contrast, the phase light or empty polymersomes collapse readily under application of minimal aspiration pressures.

5. 100 nm-Sized Polymersome Disintegration Kinetics

Subsequent to poration, growth of the membrane pores increasingly destabilizes the vesicle carrier (FIG. 16A(iii)). To gain further insight into the complete loss of membrane integrity (especially with circulation-favored 100-nm vesicles, see Photos et al., 2003), dynamic light scattering (DLS) was used to monitor 100-nm vesicle populations of either OL1 or OL2 (TABLE ⁶:f_{EO}=0.49) again blended with OB18 (at 25:75 mole ratio as above). Vesicles were first sized down to a single population of 100±20 nm by sonication, freeze thaw, and cyclic extrusion (Lee et al., 2001). As a control, the scattering intensity of a pure OB18 vesicle population was found to remain constant throughout the course of the studies. However, the OL blends show a progressive decay in intensity of the 100-nm peak. This peak increasingly splits up into two distinct populations consisting of larger fragments of aggregates (perhaps extended vesicles or worms; see FIG. 14), and a smaller peak at 40 nm that probably corresponds to micelles. The latter identification is certainly consistent with prior characterizations of PEG-PLA micelles (Yasugi et al., 1999; Kim et al., 2002; Hagan et al., 1996; Kim et al., Polymers for Advanced Technologies 10:647-654 (1999)).

From DLS, disintegration time constants for the OL1 and OL2 blended vesicles were measured to be τ_{disintegration}=12 and 4 days, respectively. The τ_{disintegration} for OL1 appeared to be several-fold longer than the τᵣₑₗₑₐₛₑ determined for the same OL composition. The DLS results are therefore consistent with post-release disintegration. It might seem surprising that similar blends with OL2 display three-fold faster vesicle disintegration kinetics than OL1, especially since the PLA block of OL2 is less than one-fourth larger in molecular weight than that of OL1 (Mₙ; TABLE 6). However, the three-fold faster disintegration together with the concomitant emergence of a micelle peak implies that the larger the f_{EO} of a diblock (as in OL2), the stronger its propensity to rapidly transform into a detergent-like moiety that tends to destabilize existing bilayer morphologies.

6. Blend-Dependent Release Kinetics

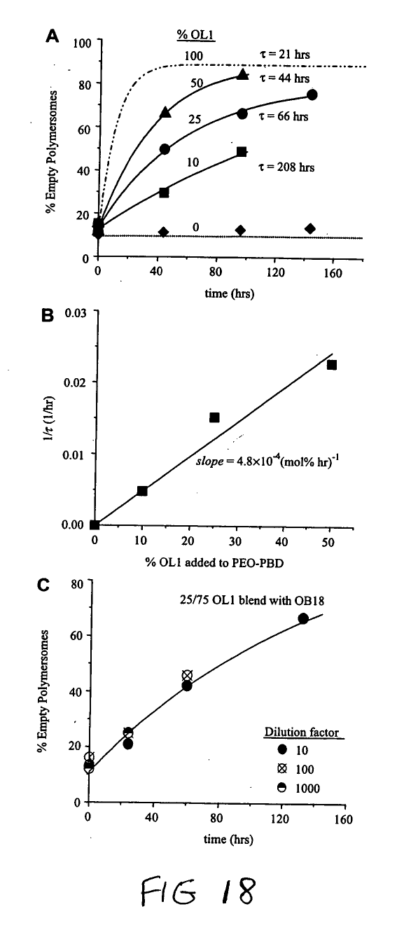

The influence of hydrolysable PEG-PLA chains on release kinetics was further elucidated and directly controlled by varying the mole fraction of OL1 blended into the OB18 membrane. At initial times, nearly all vesicles (90-100%) were loaded with hydrophilic encapsulant, irrespective of blend ratio. Depending on this ratio, the characteristic release time (τᵣₑₗₑₐₛₑ) was observed to vary from tens of hours to days (FIG. 18): this figure indicates that an increasing mole fraction of OL1 in the aggregate system accelerates encapsulant release from these giant carriers (FIG. 16: i→ii, iii).

Monitoring vesicle populations in a blend for t>τᵣₑₗₑₐₛₑ reveals a progressive disintegration of empty vesicles (see FIG. 16A(iii)). Loss of these empty vesicles results in an anomalous shift in the release curve and leads to an increase in the relative population of residual, “loaded” vesicles. Nonetheless, based on the initial observation times, the rate constant for release kᵣₑₗₑₐₛₑ=1/τᵣₑₗₑₐₛₑ is found to be a linear function of the initial mole percent of OL1 blended with inert OB18 (FIG. 18B):
kᵣₑₗₑₐₛₑ=const×[polyester]_{B} Eq (6)

Extrapolation of the plotted release kinetics to vesicles of pure (100%) OL1 (e.g., FIG. 14B) gives τᵣₑₗₑₐₛₑ˜21 hours as sketched in FIG. 18A. This time scale is short relative to vesicle formation times of τ_{formation}˜10-15 hours. It is, therefore, clear why formation of pure PEG-polyester vesicle systems has remained elusive. Furthermore, these blends clearly deepen the understanding of the degradation process by protracting the release time scales. Indeed, robust characterizations of the lower mole fraction systems are not problematic since τᵣₑₗₑₐₛₑ>>τ_{formation}.

In an effort to concomitantly infer localization of PEG-PLA in the polymersome membrane, as well as its role in facilitating encapsulant loss, release kinetics from 25:75 (mol %) blends were monitored after dilution, by up to three orders in magnitude of bulk solution. FIG. 18C demonstrates only minor deviations in the time scale of encapsulant release with such dilution (τᵣₑₗₑₐₛₑ±15%). Bulk PEG-PLA must, therefore, have no role in the process. This confirms the central importance of polyester chains pre-localized in the vesicle membrane (see, FIG. 14C) in both encapsulant release and eventual carrier destabilization. It is thus readily envisioned that for any individual vesicle, release is a burst-like, two-state process (FIG. 16). For a population of vesicles, this effect appears graded, as would be expected of a protracted first order process typified by Eq. (6).

Lastly, initial tests of vesicle poration in human plasma (and 37° C.) showed similar initial stability and release profiles as found in this example using physiological buffer.

7. Release Kinetics for PEG-PCL

To confirm a very general role for polyester hydrolysis as the ‘trigger’ for polymersome destabilization, the diblock copolymers of PEG-PCL (OCLs in TABLE 6) were also investigated. PCL, like PLA, has been widely explored as a degradable polyester (Pitt, in: Biodegradable Polymers as Drug Delivery Systems, 1990; Chawla et al., 2002; Kweon et al., Biomaterials 24:801-808 (2003)), but its six-carbon backbone makes it more hydrophobic than a PLA chain of comparable MW. When hydrated as pure diblocks, the OCL copolymers self-assemble into morphologies consistent with their respective f_{EO} fractions (see TABLE 6). For example, being near the phase boundary, OCL1 self-assembles into a mixed population of both vesicles and cylindrical or worm micelles. It is therefore not surprising that membrane blends with OCLs, and the inert OB18 form as readily as with the OL diblocks. With 25:75 molar blends of OCL in OB18, encapsulant release kinetics from micron-sized vesicles were again a function of copolymer chemistry. OCL1, as well as both OLs (OL1 and OL2), have comparable hydrophobic block molecular weights (Mₕ=3.3±0.6 kDa).

However, OCL1 has an intermediate f_{EO} (see TABLE 6). Therefore, one might naively expect OCL1-based vesicles to release faster than similar OL1 blended vesicle compositions. At the same time, OCL1-based vesicles should also release slower than blended compositions with OL2 (τᵣₑₗₑₐₛₑ=40 hours; TABLE 7). However, the release time determined for OCL1 (τᵣₑₗₑₐₛₑ=73 hours) proves to be slightly longer than that of OL1 (τᵣₑₗₑₐₛₑ=66 hours). This deviation from naive expectation provides the clearest indication of a slower hydrolysis for the more hydrophobic PCL chemistry within the membrane core.

The second PEG-PCL diblock, OCL2, has the most membrane-preferring proportions with f_{EO}=0.28. OCL2 also has a four-fold larger PCL block (Mₕ≈13 kDa). Encapsulant release from OCL2/OB18 vesicles proves to be two-fold slower in comparison with the most similarly proportioned OL1 (f_{EO}˜0.33) blends. One likely factor is that water activity in the PCL core is lower than in a PLA core. In addition, a greater degree of ester hydrolysis would be required to drive this stable bilayer-forming copolymer (f_{EO}˜0.28) into an active detergent-like molecule (f_{EO}>0.4) that then destabilizes the carrier membrane. Consequently, both f_{EO} and polyester chemistry (PCL vs. PLA) thus play a more dominant role in dictating release kinetics than molecular weight effects do. Discussion

1. Copolymer Integration into Membranes

When hydrated initially, the PEG-polyester copolymers and blends self-assemble into stable bilayer architectures (e.g., FIG. 14B). The core thickness of the PLA membrane is similar to a previously studied PEG-PBD vesicle Bermudez et al., 2002), namely EO₅₀-BD₅₅ (with d≈10.6±1 nm). This OL1 result fits the general scaling found for PBD cores of d˜N^{0.5}. While PLA has a high oxygen content, such high oxygen contents in hydrophobic blocks are not a limitation to membrane formation. At least one Pluronic triblock copolymer with an oxygen-rich midblock (EO-polypropyleneoxide-EO) has previously been reported to form vesicles (Schillen et al., Macromolecules 32:6885-6888 (1999)).

TABLE 7
Encapsulant release times or rates from pure or blended
membranes with hyrolysable block copolymers

| | | Kᵣₑₗₑₐₛₑ (×10⁴) | |
|---|---|---|---|
| | τᵣₑₗₑₐₛₑ(h) for 25:75 | (mol % in | τᵣₑₗₑₐₛₑ(h) for pure |
| Copolymer | blend with OB18 | OB18 hr)⁻¹ | copolymer^{a} |
| OL1 | 67 | 4.7 | 22 |
| OL2 | 40 | 10.1 | 0^{b} (10) |
| OCL1 | 73 | 5.5 | 0^{b} (18) |
| OCL2 | 129 | 3.1 | 32 |

^{a}τᵣₑₗₑₐₛₑ linearly extrapolated from 25% copolymer blends.
^{b}τᵣₑₗₑₐₛₑ = 0 for copolymers that cannot, when pure, form vesicles.

Membrane-localized fluorescent PLA demonstrates PEG-PLA integration (FIG. 14C). Further detailed intensity analysis of these labeled blends (FIG. 15) shows a strong linear trend as a function of the mol % added to the membrane. This proportional increase in fluorescent intensity along with NMR spectroscopy on 50:50 blends clearly shows membrane miscibility of OL in PEO-PBD. Separate evidence of mixing in blends has recently been demonstrated by free radical cross-linking of the unsaturated polybutadiene (PBD) double bonds in OB18 (Ahmed et al., 2003). Cross-linking effectively blocks lateral mobility of the PBD chains in the bilayer architecture. Extraction of the blended OL1 chains by chloroform leads to rapid encapsulant release (in minutes) and the consequential loss of membrane integrity. In contrast, cross-linked shells of pure OB18 prove extremely robust and unaffected by external chemical and physical stresses (Discher et al., J. Physical Chemistry B 106:2848-2854 (2002)).

2. Release Kinetics of Hydrophilic Encapsulants

Much of the previous work on PEG-PLA based aggregates can be categorized as assemblies of copolymers with low f_{EO} and large molecular weight PLA blocks, (a “crew-cut” presentation of PEG per Eisenberg et al. (see Allen et al., 2000)), or else copolymers with f_{EO}>0.4. Depending on the nature of aggregate processing, the former generally leads to the sequestering of glassy, immobile PLA blocks into solid-like particles, whereas the latter leads to an assembly of micellar structures as per FIG. 14. Only lipophilic compounds can be intercalated into such diblock morphologies. Micellar aggregates give release profiles that correlate with progressive PLA degradation on the order of weeks (Matsumoto et al., 1999; Piskin et al., 1995) to months (Kostanski et al., Pharmaceutical Development and Technology 5:585-596 (2000)).

Particulate systems, on the other hand, display distinct biphasic burst profiles with repartitioning and leakage of a lipophilic drug varying from minutes to tens of hours (Gref et al., 1994; Avgoustakis et al., 2002). A critical issue with PEG-PLA delivery systems is burst (Matsumoto et al., 1999; Avgoustakis et al., 2002; Li et al., Pharmaceutical Research 18:117-124 (2001)), as opposed to progressive degradation (Piskin et al., 1995) release profile. Efforts have been made to suppress or rather “soften” this burst release by coating aggregates with proteins, amphiphiles, or polymers, such as albumin (Araki et al., Artificial Organs 23:161-168 (1999)), poloxamers (Morita et al., Europ. J. Pharmaceutics and Biopharmaceutics 51:45-53 (2001)), or detergents (Matsumoto et al., 1999). However, in the present examples, membrane blends of an inert copolymer plus PEG-PLA have succeeded not only in self-assembling into stable vesicles for hydrophilic encapsulant release, but also in providing uniquely tunable release times (τᵣₑₗₑₐₛₑ=hours to days) that depend linearly on the blend ratio of PEG-PLA. Additionally, the lack of dependence of τᵣₑₗₑₐₛₑ on dilution of the vesicles (FIG. 18C) excludes any possible role of external copolymer (i.e., OL1) in vesicle poration.

Polymer vesicles change shape by swelling and shrinking osmotically (Discher et al., 1999), indicating that water permeates the core of the membrane. Such water can also initiate hydrolytic cleavage of the PLA or PCL blocks sequestered within the core. Considerable work has already been done on the mechanism of this water-initiated reaction (e.g., Schmitt et al., Macromolecules 27:743-748 (1994)), and it is well under-stood that the degradation of large molecular weight PLA blocks, self-assembled as either micelle or nano-particles, takes on the order of months (Gref et al, 1994). However, the presence of hydrophilic PEG, either through attachment (Shah et al., J. Biomaterials Science, Polymer Ed. 5:421-431 (1994); Li et al., J. Appl. Polymer Science 78:140-148 (2000)), or blending (Jiang et al., Pharmaceutical Research 18:878-885 (2001)) is claimed to direct the uptake of water, leading to accelerated (15-fold) dissolution kinetics (Penco et al., Biomaterials 17:1583-1590 (1996)).

3. Hydrolysis-Driven Membrane Poration

In general, controlled release occurs as a result of poration by PLA or PCL hydrolysis in the diblock copolymer membrane. The aqueous water microenvironment facilitates ester hydrolysis either by chain-end (Belbella et al., 1996; Shah et al., 1994), and/or random (Belbella et al., 1996; Jellinek, Aspects of Degradation and Stabilization of Polymers, Elsevier, New York, pp. 617-657 (1978)) scission in the core of the membrane, or at the PEG-polyester interface. If the latter interfacial degradation were dominant, the intact polyester block would simply sequester within the richly hydrophobic core of the membrane, and create inclusions (not seen) while PEG diffuses away. In contrast, other mechanisms of PEG-polyester degradation eventually porate the vesicles.

The time constant found for characteristic release from 50:50 blends with OL1/OB18 (τᵣₑₗₑₐₛₑ=44 hours) has been shown, with particles composed of similar PEO-PLA blocks (f_{EO}˜0.33), to liberate ˜50% of the lactic acid (Avgoustakis et al., 2002). Langer et al. also studied similar particles and observed analogous release kinetics within an hour, but with essentially ˜0% lactic acid generation (see, Peracchia et al., J. Controlled Release 46:223-231 (1997)). It can be implied from such previous experiments that only a small fraction of the blended polyesters is required to trigger the controlled destabilization of the vesicle carriers, consistent with the findings by the inventors.

The onset of hydrolysis and resultant curvature preference of OL1 chains in the membrane of a vesicle transforms this stable bilayer-forming chain into a detergent-like copolymer. Such degraded chains with comparatively short hydrophobic blocks will tend to segregate from their inert, entangled OB18 neighbors (Lee et al., 2001), congregate and perturb local bilayer curvature, and ultimately induce hydrophilic (i.e., PEG-lined) pores in the membrane. These salient molecular scale transitions are evident in physical observations, such as molecular weight-dependent encapsulant release from otherwise intact vesicle carriers (FIG. 18). Liposomal systems have applied similar principles, such as doping non-reactive amphiphiles with reactive ones (Rui et al., J. Amer. Chem. Soc. 120:11213-11218 (1998)) to exploit molecular scale transitions from lamellar to “non-bilayer” forming chains (Adlakha-Hutcheon et al., 1999; Needham et al., Advanced Drug Delivery Rev. 53:285-305 (2001)) or to inverted hexagonal phases (Holland et al., 1996; Zhigaltsev et al., 2002; Guo et al., 2003) in order to concomitantly trigger encapsulant release and carrier destabilization.

To further verify the evolution of OL1 chains into detergent-like triggers, pure encapsulant loaded OB18 vesicles were incubated with exogenous OL1 block copolymer in the aqueous bulk solution. Over time, the surface active OL1 chains increase (inert) vesicle permeability, and trigger the release of hydrophilic encapsulants (data not shown). Though OL activity appears to be analogous to detergent-mediated solubilization of vesicle membrane, the dissolution kinetics were three orders of magnitude slower than TX-100 solubilization of micron-sized OB18 vesicles (Pata et al., Langmuir (2004) (submitted for publication)). This delay in vesicle instability parallels work by Ladaviere et al. on liposome destabilization by amphiphilic macromolecules (Ladaviere et al., J. Colloid Interface Science 241:178-187 (2001); Ladaviere et al., Langmuir 18:7320-7327 (2002)).

At least two distinctions are noteworthy. First, liposomal assemblies invariably lack the dense 100% PEGylated “hairy” brush that deters adsorption and integration of factors that limit vesicle circulation times in vivo (Photos et al., 2003). Second, the ability of amphiphilic polymers to modulate membrane properties is conditional on the hydrophobicity of the adsorbing polymer. In the present case, the oxygen-rich PLA block handicaps the polymer and renders it a weak, but adequate solubilizer. In particular, partially degraded polyester chains are responsible for curvature by minimizing the membrane line tension around pores, while also leading to the slow growth of pores in the otherwise impenetrable membrane. However, the molecular weight-dependent release profiles of hydrophilic dextrans from polymersomes (see FIG. 17) indicate stable pore sizes that approximate the membrane's thickness.

As to whether amphiphilic polymers exhibit self-healing tendencies in vesicle pores, it was determined that steric hindrance due to chain repulsion arises with the hairy PEG brush that lines the pore in the bilayer membrane, thereby deterring membrane resealing. Regardless of why this occurs, PEG-polyester chains in bilayer morphology are poised to act as time-evolving molecular triggers that modulate encapsulant release and subsequent vesicle disintegration.

4. Microphase Basis for Poration Kinetics

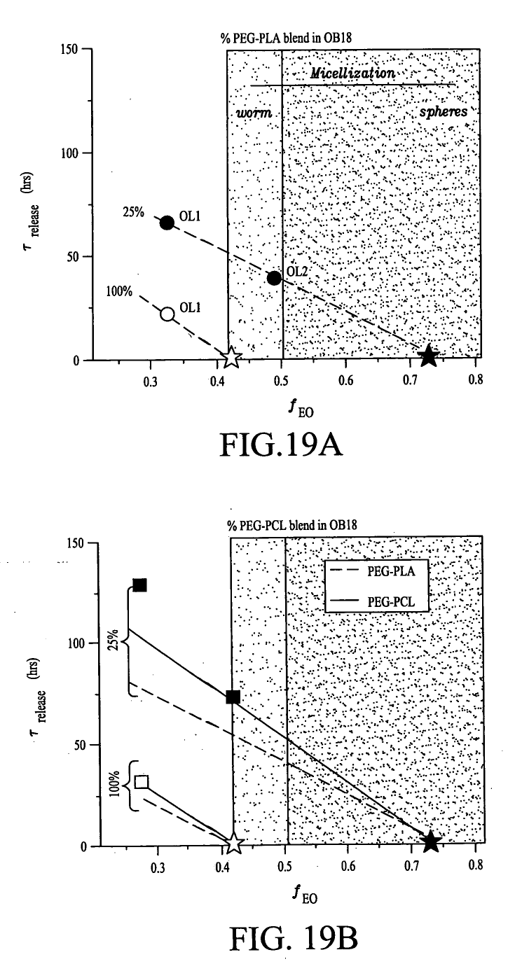

The phase boundaries indicated in FIG. 14 provide a framework for graphically understanding encapsulant release times as a function of the key variable f_{EO}. Considering first the two PEG-PLAs that were studied (FIG. 19A), the small difference in molecualr weight of the hydrophobic block (Mₕ) was neglected and a single line was drawn through the two data points for 25% blends. The f_{EO} intercept of this first line (black filled star: polyester diblock f_{EO}=0.73) indicates a blended OL/OB18 system (25:75), which would provide instant release upon vesicle formation. A nearly parallel line was also sketched through the result for 100% OL1, but this second line intersected the τᵣₑₗₑₐₛₑ=0 axis at f_{EO}=0.42 (open star). This intercept is again indicative of a system displaying instant release and dominant micelle formation, as opposed to any significant vesicle-delayed encapsulant release.

Similar conclusions were drawn from the PEG-PCL systems (at 25 mol %) plotted in FIG. 19B. While the baseline release from pure (100%) vesicles is theoretically important, both ‘star’ systems are impractical for release applications, since high vesicle yields by standard hydration methods take a comparatively long formation time, as explained above.

Though the over-simplifications here do not fully address nuances of co-existence between vesicle/worm/sphere regimes found experimentally, such as those illustrated in FIG. 14B, the various lines on the two plots of FIG. 19 are assumed to be representative of release times for the three smaller block copolymers studied here (i.e., OL1, OL2, and OCL1). Lastly, for the larger OCL diblock, OCL2, the two square points off the lines in FIG. 199B highlight relatively small offsets (<25%), despite a ˜4-fold larger hydrophobic block. Small offsets imply a minimal influence of Mₕ on release kinetics in comparison to the strong effects of the initial f_{EO} of a copolymer. This conclusion is fully consistent with the assertion here that ±20% differences in Mₕ of the three smallest diblocks (i.e., OL1, OL2, OCL1: 3.3±0.6 kDa) are simply insignificant to release kinetics.

Within the framework of microphase behavior, the moderate molecular weight polyester-based diblocks, such as the OL and OCL, self-assemble or integrate into bilayer architectures that are sensitized for release. Triggered by the initiation of hydrolysis in the core of the membrane, the onset of pores with highly curved edges leads to the observed release of lumenal encapsulants. Eventually, these vesicle carriers disintegrate into mixed micellar assemblies of worms and spheres. Polyester participation in the bilayer morphology appears to be strongly conditional on the rate of hydrolysis of the hydrophobic block (e.g., PCL vs. PLA) as well as the hydrophilic block ratio (f_{EO}). Another important means of controlling release involves the formation of blends of degradable polyesters with inert diblocks (e.g., f_{EO}<0.73 for 25:75 blends), and its stable integration into a mixed membrane. In contrast, for pure (100%) polyesters, extrapolations prove relatively independent of hydrophobic block chemistry and allow vesicle formation and release within f_{EO}<0.42.

In sum, The kinetics of hydrolytically triggered destabilization of polymersomes composed or blended with degradable PEG-PLA or PEG-PCL and the inert PEG-PBD (OB18) have been elucidated by sucrose and fluorophore leakage assays for giant vesicles as well as DLS of nanovesicles. Labeling of the PLA block demonstrates the participation of the polyester chain in stable membrane integration. Subsequent polyester hydrolysis in the core of the membrane transforms these bilayer-forming chains into active, detergent-like moieties that trigger the induction of pores in the vesicle membrane. Leakage of hydrophilic encapsulants occurs in a first-order, degradation-dependent fashion on time scales ranging from hours to tens of days. Molecular-weight-dependent encapsulant release assays determine the finite pore size to be comparable to the thickness of the vesicle membrane (˜10 nm).

Parallel studies with varied polyester hydrophilic/hydrophobic block ratios, hydrophobic core chemistry, and different mole percent blends indicates that polyester chain hydrolysis is the molecular trigger controlling encapsulant release and carrier destabilization kinetics. In other words, the controlled rate of release of the encapsulant, e.g., the dyes and/or drugs etc, from the hydrolysis-triggered controlled release polymersome vesicles of the present invention is controlled by the blend ratio of the copolymers, copolymer molecular weights, and/or copolymer block ratios (i.e., the weight fraction, f_{EO} of the polyethylene oxide or PEG moiety).

Additional features of this potential drug delivery system include the 100% PEGylated brush that has been demonstrated elsewhere to effectively deter opsonization and prolong nano-sized vesicle circulation. Polyester chains play a crucial role in conferring release mechanisms as well as definitive biocompatibility. Salient features of these polymersomes include resistance to destabilizing agents, such as phospholipases and other lipid-disruptive components. The thick hydrophobic core of the vesicle membrane enhances loading efficiencies of lipophilic drugs. Thus, the present invention is useful because it further enables the study release and delivery of synergistically active lipophilic and hydrophilic drugs from these parent systems, since transitions from the bilayer to micellar regime may provide a sustained depot for lipophilic drug and impart novel pharmokinetics.

### Example 6

### Drug Delivery Via Degradable Polymersomes: Mechanistic Aspects of Uptake, Release, and Cytotoxicity of Hydrophilic and Hydrophobic Encapsulants

Degradable polymersomes are vesicle carriers containing hydrolysable block copolymers. To confirm the effectiveness of the hydrolysis-triggered controlled release mechanism on either hydrophilic or hydrophobic encapsulants from the self-porating polymersomes, and to confirm delivery to targeted cells, which is essential to drug delivery in a patient, both types of drugs were loaded and tested to determine their release into human cells. Drug loading efficiencies and in vitro release from polymersomes were monitored by fluorescence methods, as above.

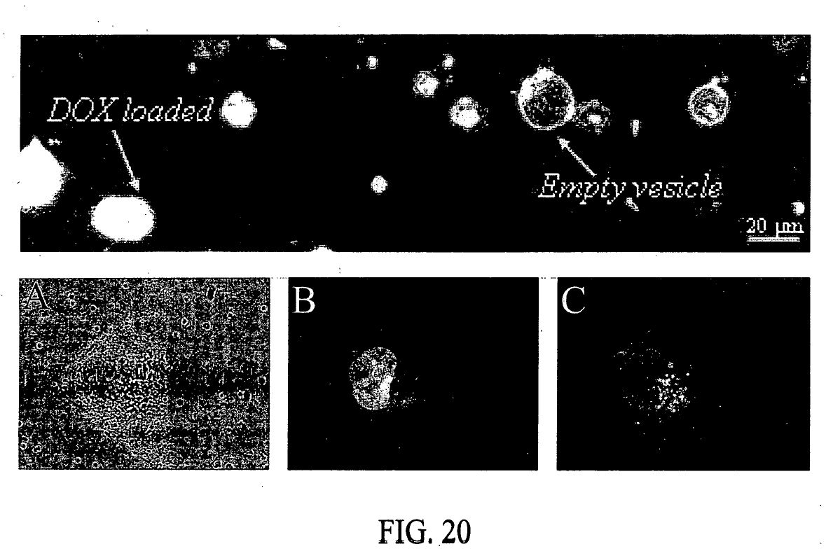

Experiment 1: Using a hydrophilic encapsulant. For the present cell studies, vesicles of 25 mol % blends of OL2 in OB18 (75%) were loaded with the hydrophilic anticancer drug, doxorubicin (a cytotoxic anthracyclin as used in Example 5), bearing a fluorescent marker (fluorescent red DOX) using a well established pH gradient method, and labeled green with a hydrophobic membrane dye. Dual labeling of the polymersome carrier allows a visual confirmation of “loaded” drug (yellow as a result of red and green fluorescence overlay) or else “empty” (green) vesicles over the time course of an experiment either in vitro or within cells (shown in black and white in FIG. 20A). The fluorescent images of degradable polymersome carriers showed them to be loaded with the anticancer drug, doxorubicin (DOX) (FIG. 20A).

FITC-labeled, DOX-loaded degradable polymersomes (23 μg DOX/mg polymer) were incubated for 4 hour at 37° C. with MDA-MB231 (human breast cancer epithelial cells), and showed uptake within hours by passive endocytosis (FIG. 20). Nuclear delivery and in vitro release from the degradable polymersome carrier loaded with DOX were studied by nuclear fluorescence and by a methyl thiazole tetrazolium (MTT) viability assay. Doxorubicin localization to the nucleus and in vitro cytotoxicity were respectively demonstrated as seen in overlays of bright field and fluorescent images showing nuclear localization of DOX (recognized as a red emission) and perinuclear localization of the associated polymersomes (recognized as a green emission) (visible to the extent possible in black and white in FIGS. 20C and 20D).

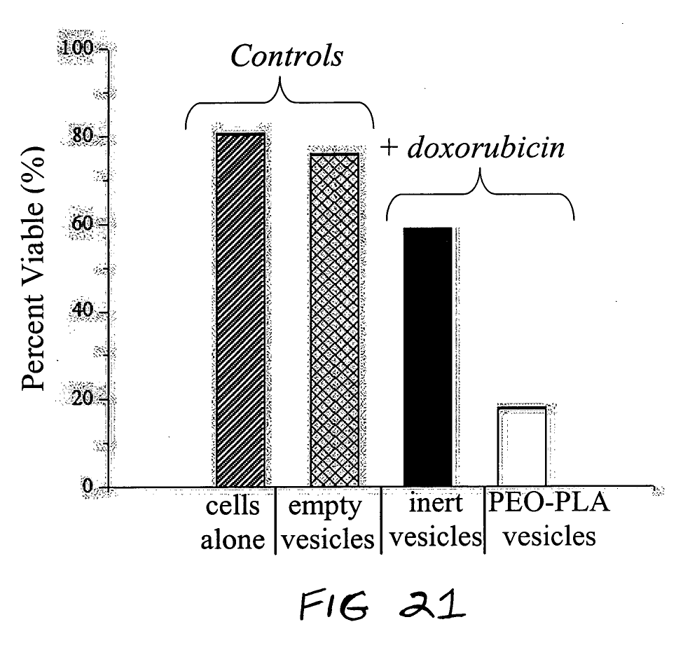

Cytotoxicity assay of the MDA-MB231 cells treated with DOX-loaded degradable polymersomes (OL2/OB18 blended at 25:75 mol % ratio) showed effective delivery (FIG. 21). MDA-MB231 cells were incubated with 0.3 mg/mL DOX associated with polymersomes for 2 hours before being washed, and subsequently analyzed by the standard MTT assay at 24 hours. As shown in FIG. 21, unloaded, empty polymersomes were utilized as controls. Inert, non-degradable polymersomes showed a slight leak of DOX based on a small cytotoxic effect of the drug.

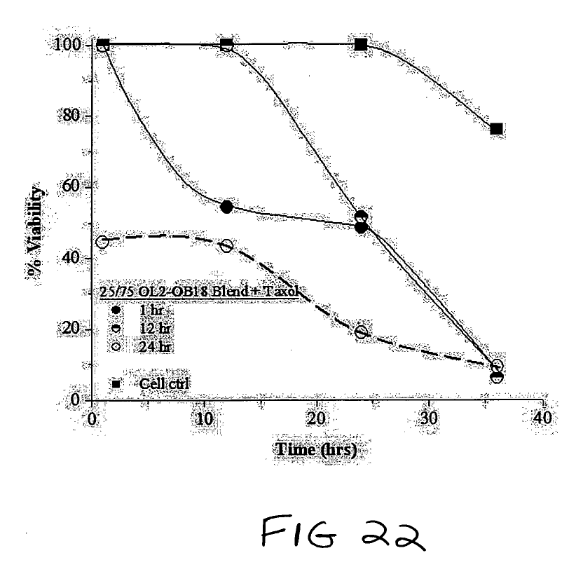

Experiment 2: Using a hydrophobic encapsulant. Taxol, a second, common anti-cancer drug was also studied by similar means used above, and with similar results (FIG. 22). However, taxol is a hydrophobic drug sequestered by intercalation into the membrane, rather than retained as doxorubicin is, within the lumen core of the polymersome. Additionally, mechanistic aspects of intracellular drug release have been demonstrated by showing surfactant-like lytic activity against cell membranes with the degradable polymer above critical concentrations.

To show that taxol-loaded polymersomes accumulate in cells at early time points, degradable polymer vesicles of OL2/OB18 blends (25:75 mol % ratio, as above) were loaded with FITC-labeled drug, taxol (43 ng drug/mg polymer) in an aqueous phase, after vesicle formation. The drug loaded carriers were sized down to ˜100 nm by sonication, freeze-thaw, and extrusion, as described above. Excess, non-encapsulated drug was removed by dialysis (MWCO 1 MDa). The taxol-loaded vesicles were incubated with the MDA-MB231 cells for either 1 or 4 hours, respectively, and fluorescence microscopy images showed rapid taxol labeling of the polymersome membranes. Internalization and perinuclear localization of the drug-loaded vesicles was consistent with taxol being a hydrophobic drug.

Moreover, cell proliferation was inhibited in the presence of the taxol-loaded degradable polymersomes. This was seen when the cells were incubated for 1, 12, and 24 hours, respectively, with 120 ng/mL taxol intercalated into polymersomes comprising 25 mol % blends of OL2 in 75 mol % OB18. After the initial exposure, the cells were washed and subsequently analyzed by the MTT assay at the desired times, at points ranging from 0 to 36 hours (FIG. 22).

Notably, additional data indicated that refrigeration storage of the polymersomes using a PEO-PCL copolymer, at 4° C., reduced degradation to near zero over a period of at least 3 weeks. This clearly shows that the release observed of the encapsulant from the polymersome carrier was the result of an active process (hydrolysis-triggered poration), not simply first degree kinetics involved with gradual seepage over time from the intact membrane.

In sum, the results presented and the accompanying figures shown above, using cancer cells, as representative human cell targets, showed that degradable polymersomes effectively deliver both hydrophilic and hydrophobic encapsulants to cell targets as proposed. The hydrophilic doxorubicin was both loaded and subsequently released from the vesicle lumen in a controlled release manner, ultimately killing the cells; then taxol, being hydrophobic, was loaded and subsequently released from the vesicle membrane, also killing the cells. Thus, PEG-polyester chains in bilayer morphology are poised to act as time-evolving molecular triggers that modulate hydrophilic or hydrophobic encapsulant release when delivered to a cell, either in vitro or when delivered to the cells of a patient in vivo.

All patents, patent applications and publications referred to in the present specification are also fully incorporated by reference.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the basic principles of the invention. Such modifications and additional embodiments are also intended to fall within the scope of the appended claims.

1. A method of preparing stable, purely synthetic, self-assembling, controlled release, polyethylene oxide (PEO)-based polymersome vesicles having a semi-permeable, thin-walled, amphiphilic, high molecular weight PEO-based block copolymer encapsulating membrane and at least one active agent encapsulated therein, said method comprising:
determining the appropriate blend ratio (mol %) of hydrolysable PEO-block copolymer of at least one hydrophilic component and at least one more hydrophobic PEO-block copolymer component to produce amphiphilic high molecular weight PEO-based polymersomes having a desired controlled release rate of the encapsulated encapsulant;
selecting the at least one hydrolytically degradable, hydrophobic block copolymer to effect controlled polyester chain hydrolysis in the membrane, such that when combined with hydrophilic PEO, the PEO volume fraction (f_{EO}) and chain chemistry control encapsulant release kinetics from the copolymer vesicles and polymersome carrier membrane destabilization; and
blending in aqueous solution said at least one hydrophilic PEO-block copolymer together with the at least one inert, hydrophobic PEG-block copolymer to produce amphiphilic high molecular weight PEO-based polymersomes having the desired controlled release rate of the at least one encapsulant contained therein.

2. The method of claim 1, wherein the polyethylene oxide component of the block copolymer is polyethylene glycol (PEG), or structural equivalent thereof.

3. The method of claim 2, wherein the at least one hydrophilic block copolymer comprises a block copolymer of PEG and a hydrolytically degradable polyester.

4. The method of claim 3, wherein the hydrolytically degradable polyester comprises a high molecular weight polyester of polylactic acid (PLA), which when combined with PEG forms PEG-PLA, or a high molecular weight polycaprolactone (PCL), which when combined with PEG forms PEG-PCL.

5. The method of claim 1, wherein the at least one inert, non-hydrophilic block copolymer comprises polybutadiene.

6. The method of claim 1, further comprising increasing the mole fraction (mol %) of the at least one hydrolytically degradable block blended into the inert copolymer to directly control release of the encapsulant upon subsequent hydration.

7. The method of claim 6, wherein increasing the block f_{EO} increases rate of transformation into a detergent-like moiety, thereby accelerating destabilization of bilayer morphology of the polymersome membrane and encapsulant release.

8. The method of claim 1, further comprising selecting the at least one polyester for biocompatibility.

9. The method of claim 1, wherein the at least one encapsulant is an amphiphilic or lipophilic composition.

10. The method of claim 1, wherein the at least one encapsulant ranges in molecular weight from less than 10² Da to more than 10⁵ Da.

11. The method of claim 1, wherein increasing molecular weight of the at least one encapsulant decelerates rate of release from the polymersome carrier, but the f_{EO} and polyester selection primarily dictate release kinetics.

12. The method of claim 9, wherein the at least one encapsulant is a hydrophilic encapsulant encapsulated in the lumen of the polymersome, or the at least one encapsulant is a hydrophilic encapsulant encapsulated by intercalation into the polymersome membrane, or there is more than more encapsulant selected from one or more hydrophilic encapsulants or one or more hydrophobic encapsulants, or a combination thereof.

13. The method of claim 12, wherein at least one hydrophilic encapsulant is selected from the group consisting of carbohydrates, including sucrose; marker-tagged dextrans, including fluorescent dextrans from 1 kD up to 200 kD; therapeutic compositions, including doxorubicin or amphoterican B; dyes; indicators; protein or protein fragments, including catalase; ammonium sulfate; salts; and gene or gene fragments, including oligonucleotides.

14. The method of claim 12, wherein at least one hydrophobic encapsulant is selected from the group consisting of PKH fluorescent dyes; therapeutic compositions, including taxol and anthracyclin; monosialoganglioside; fluorinated lipids; fluorescein-taxol; and fluorescent-dye modified copolymers.

15. The method of claim 12, wherein the at least one therapeutic composition is an anti-cancer drug selected from cytotoxic doxorubicin and taxol.

16. The method of claim 1, wherein the at least one encapsulant is encapsulated simultaneously with polymersome formation, or subsequent thereto.

17. The hydrolysis triggered, controlled release polymersome produced by the method of claim 1.

18. A method of releasing at least one encapsulant from the polymersome vesicle prepared by the method of claim 1 to an environment immediately surrounding the polymersome, wherein the method comprises:
delivering the polymersome and said at least one encapsulant contained therein to an intended environment, wherein the composition of the environment triggers polyester hydrolysis at a predetermined rate in the polymersome membrane;
transforming membrane bilayer chains into active detergent-like moieties;
triggering induction of pores in the membrane; and thereby effecting release of said encapsulant.

19. The method of claim 18, wherein said method of release further comprises administering the polymersome to a patient, and releasing said encapsulant from the polymersome to said patient, wherein the polymersome and encapsulant are biocompatible.

20. A method of self-removal of the controlled release polymersome delivered to the patient in claim 19 following release of the encapsulant, comprising further inducing poralation of the membrane to continue until the polymersome is disintegrated.
